(19) United States
(12) Patent Application Publication
(10) Pub. No.: US 2026/0256907 A1
(43) Pub. Date: Sep. 3, 2026

(54) OLIGONUCLEOTIDES FOR USE IN MODULATING IMMUNE RESPONSES AGAINST HEPATITIS B VIRAL INFECTION

(71) Applicant: AUSPERBIO THERAPEUTICS INC., Foster City, CA (US)

(72) Inventors: Guofeng CHENG, Foster City, CA (US); Cheng Yong YANG, Foster City, CA (US)

(21) Appl. No.: 19/366,428

(22) Filed: Oct. 22, 2025

Related U.S. Application Data
(63) Continuation of application No. PCT/CN2024/089195, filed on Apr. 22, 2024.

(30) Foreign Application Priority Data
Apr. 23, 2023 (WO) PCT/CN2023/090098

(51) Int. Cl. A61K 39/29; A61K 39/00; A61P 31/20; C12N 7/00

(52) U.S. Cl. CPC A61K 39/292; A61P 31/20; C12N 7/00; A61K 2039/53; A61K 2039/55505; A61K 2039/55561; C12N 2730/10134

(57) ABSTRACT

Provided herein are vaccine compositions comprising an oligonucleotide and a Hepatitis B viral antigen, either present in the same composition, or in separate compositions, useful for prevention or treatment of Hepatitis B viral infection in a subject. The vaccine compositions can exert a sustained immunomodulatory effect and lead to the reduction of HBV antigen levels in an infected subject. Also provided herein are methods of making and use thereof.

### RELATED APPLICATIONS

This application is a Continuation of PCT/CN2024/089195, filed on Apr. 22, 2024, which claims the priority and benefit of PCT Application No. PCT/CN2023/090098, filed on Apr. 23, 2023, the contents of which are incorporated herein in its entirety.

### REFERENCE TO AN ELECTRONIC SEQUENCE LISTING

The contents of the electronic sequence listing (AUSB_005_01US_SeqList_ST26.xml; Size: 6,545,585 bytes; and Date of Creation: Oct. 17, 2025) are herein incorporated by reference in its entirety.

### BACKGROUND

Hepatitis B is a viral infection that attacks primarily the liver, and can cause both acute and chronic disease. The Hepatitis B virus (HBV) transmitted parenterally by contaminated material such as blood and blood products, contaminated needles, sexually, and vertically from infected or carrier mothers to their offspring. The World Health Organization estimates that more than 2 billion people have been infected worldwide, with about 296 million people living with chronic hepatitis B infection in 2019. In 2019, hepatitis B resulted in an estimated 820 000 deaths, mostly from cirrhosis and hepatocellular carcinoma (primary liver cancer). HBV is a main cause of death globally (World Health Organization Fact Sheets, Hepatitis B, Jun. 24, 2022).

HBV is a double-stranded hepatotropic virus which infects only humans and non-human primates. Viral replication takes place predominantly in the liver and, to a lesser extent, in the kidneys, pancreas, bone marrow and spleen (Hepatitis B virus biology. Microbiol Mol Biol Rev. 64:2000; 51-68.). Prophylactic vaccines are administered to healthy individuals to prevent a disease. Although there several prophylactic vaccines have been developed in order to prevent hepatitis B infection, e.g. Engerix-B (SmithKline Beecham), Recombivax HB (Merck), Heplisav-B (Dynavax), and GenHevac B (Sanofi Pasteur) face certain limitations, for example their ability to induce sufficient immune responses. Even more challenging has been the development of a therapeutic vaccine for the treatment of a Hepatitis B viral infection. Generally, a goal of therapeutic vaccines is to stimulate the patient's own adaptive immune system against specific viral antigens to destroy them. The goal in such a context would be to improve the reactivity of T cells specific to Hepatitis B, for example in those Hepatitis B virus-infected patients who have been chronically unwell. To date, no HBV vaccine has been shown to have therapeutic potential to reduce, eliminate, or cure Hepatitis B viral infection as they do not elicit sufficient immune responses in HBV patients.

Effective prevention and long-term treatment for Hepatitis B viral (HBV) infection, using vaccines, remains a challenge and unmet need. Provided herein are compositions and methods that address this need.

### SUMMARY

In one aspect, provided herein is a hepatitis B virus (HBV) vaccine composition comprising at least one oligonucleotide and at least one HBV antigen.

In another aspect, provided herein a method of treating or preventing a HBV infection, or a HBV-related disease, disorder, or condition in a subject with a vaccine, comprising administering to the subject the vaccine composition comprising at least one oligonucleotide and at least one HBV antigen.

In yet another aspect, provided herein a method of treating or preventing an HBV infection, or a HBV-related disease, disorder, or condition in a subject with a vaccine treatment, comprising administering to the subject at least one oligonucleotide and a at least one hepatitis B virus (HBV) antigen.

In other aspects, provided herein are kits and pharmaceutical compositions.

### BRIEF DESCRIPTION OF THE DRAWINGS

FIG. 1 provides a legend for the nucleoside modifications at each position of the sequences shown in FIG. 2. The column entitled “Examples” provides non-limiting examples for each type of nucleoside modification.

FIG. 2 shows a table of exemplary modified oligonucleotides of the disclosure. The modifications at each position of the modified oligonucleotide sequences are read using the legend in FIG. 1. AUS1010 to AUS1714 (SEQ ID NO: 11 to SEQ ID NO: 666) represent the modified oligonucleotide sequences. AUS1233 (SEQ ID NO: 10), also referred to as AUS1138, is a reference modified oligonucleotide sequence.

FIG. 3 shows the immune enhancing effects of selected oligonucloetides of the disclosure. The figure shows the Hepatitis B Surface antigen protein (HBsAg) levels in HDI-HBV mice (HBV animal model), following dosing with selected oligonucleotides of the disclosure.

FIG. 4 shows the immune enhancing effects of selected oligonucloetides of the disclosure. The figure shows the serum Hepatitis B Surface antigen protein antibody (HBsAb) levels in HDI-HBV mice, following dosing with selected oligonucleotides of the disclosure.

FIG. 5 shows the immune enhancing effects of selected oligonucloetides of the disclosure. The figure shows the serum HBsAg levels in HDI-HBV mice, following dosing with selected oligonucleotides of the disclosure.

FIG. 6 shows the immune enhancing effects of selected oligonucloetides of the disclosure. The figure shows the serum HBsAb levels in HDI-HBV mice, following dosing with selected oligonucleotides of the disclosure.

FIG. 7 shows the immune enhancing effects of selected oligonucloetides of the disclosure. The figure shows that normal animals exhibited an increase in serum HBsAb levels, following dosing with selected oligonucleotides of the disclosure. The results showed that the immune enhancing activity of the oligonucleotides tested was similar to or better than that of other known adjuvants in normal mice.

FIG. 8 shows that dosing with a combination of an oligonucleotide of the disclosure and HBsAg resulted in significantly stronger and sustained reduction in serum HBsAg levels and induced much higher levels of HBsAb compared to the HBsAg antigen alone.

FIG. 9 shows the effects of oligonucleotides of the disclosure on serum HBsAg and HBsAb levels in HDI-HBV mice, who have not received a pretreatment dose, and only received a vaccine dose. The data indicate that the inclusion of a pretreatment dose is optional.

FIG. 10 shows the beneficial effect of oligonucleotides of the disclosure on the serum HBsAg and HBsAb levels, T-cell response, and HBV mRNA and HBV DNA expression levels in the liver.

FIG. 11 shows the immune enhancing effects of oliognucleotides of the disclosure in a primate model, to induce anti-HBsAg antibody response in monkeys. The results indicate that a subcutaneous dosing route can activate the immune response faster and produce higher levels of HBsAb.

FIG. 12 shows that the serum HBsAb induction kinetics in mice, following dosing by oligonucleotides of the disclosure, are different than CpG1018, a known adjuvant.

FIG. 13 shows the effects of premixing of oligonucleotides of the disclosure with HBsAg vs. separate adjacent administration on HBsAb levels. The results indicate that both methods produce eventual comparable levels of HBsAb.

FIG. 14 shows that the immune enhancing activity of the oligonucleotides does not depend on complementarity to a portion of the nucleic acid sequence of a HBV genome.

FIG. 15 shows a graph showing the effect of the oligonucleotide AUS1476 and/or alum-adjuvanted recombinant small HBsAg administration on HBsAg levels in AAV-HBV transgenic mice. In FIG. 15, alum-adjuvanted recombinant small HBsAg is referred to as “vaccine.” Saline was administered as a control. The data are presented as mean±standard deviation. The arrows along the x-axis show the days that AUS1476 or alum-adjuvanted recombinant small HBsAg were administered to the mice. The limit of quantification (LOQ) of 1 IU/ml is indicated with a dotted line.

FIG. 16 shows a graph showing the effect of the oligonucleotide AUS1476 and/or alum-adjuvanted recombinant small HBsAg administration on HBsAb levels in AAV-HBV transgenic mice. In FIG. 16, alum-adjuvanted recombinant small HBsAg is referred to as “vaccine.” Saline was administered as a control. The bars show geometric means for each sample. The limit of quantification (LOQ) of 5 mIU/ml is indicated with a dotted line.

### DETAILED DESCRIPTION

It is to be understood that both the foregoing general description and the following detailed description are exemplary and explanatory only and are not restrictive of the inventions, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### I. Oligonucleotide-Containing Vaccines

Provided herein are oligonucleotides that exhibit immune enhancing effects, which, when administered in combination with an antigen (e.g. a viral antigen, e.g. a HBV antigen), are useful as vaccines. In some embodiments, the use is in a prophylactic context, and the vaccine is a prophylactic vaccine, prior to the onset of an infection. In other embodiments, the use is in a treatment context, and the vaccine is used as a therapeutic vaccine, following the onset of an infection. Accordingly, in some embodiments the oligonucleotide/HBV antigen vaccine of the disclosure is a prophylactic vaccine, and in other embodiments, the vaccine is a therapeutic vaccine.

In the therapeutic vaccine context, the inventors have surprisingly found that the oligonucleotides of the disclosure exhibit immune-enhancing effects, and can be used in a vaccine for treatment of an infection (e.g. a viral infection) when administered in conjunction with an antigen, e.g. in conjunction with a HBV antigen, for the treatment of a HBV infection in a subject. In this context, the inventors have found that oligonucleotides of the disclosure possess adjuvant-like properties and when administered in conjunction with a HBV antigen, leading to sustained reductions in viral load, as assessed by decreased HBV antigen levels, and also which is accompanied by increases in HBV-specific antibodies, decreases in HBV DNA, decreases in HBV mRNA, and/or increases HBV-specific T cell immune responses.

As contemplated herein a vaccine of the disclosure comprises at least one oligonucleotide of the disclosure and at least one antigen; likewise, a HBV vaccine of the disclosure comprises at least one oligonucleotide of the disclosure and at least one HBV antigen. In some embodiments, the at least one oligonucleotide and the at least one HBV antigen are present in a single composition and are administered together in the single composition. In other embodiments, the at least one oligonucleotide and the at least one HBV antigen are present in separate compositions, and are mixed together, prior to administration. In yet other embodiments, the at least one oligonucleotide and the at least one HBV antigen are present in separate compositions and are administered separately, e.g. sequentially or simultaneously, e.g. to the same site or different sites. For clarity, it is noted that the term “vaccine” as used herein refers to both the single composition example, and the separate composition example.

a. Oligonucleotides

The vaccines of the disclosure comprise at least one oligonucleotide and at least one HBV antigen, the two components which can be administered in separate compositions, or together in a single composition.

In some embodiments, the oligonucleotide is a single stranded oligonucleotide. In some embodiments, the oligonucleotide is a double stranded oligonucleotide, or has portions that are double stranded. In some embodiments, the oligonucleotide is a stranded oligonucleotide, that under certain conditions, may form double stranded secondary structures.

The length of the oligonucleotides of the disclosure can be from about 10 to about 60 nucleobases in length. In some embodiments, the oligonucleotide is about 20 to about 50 nucleobases, about 20 to about 40 nucleobases, about 20 to about 30 nucleobases, about 15 to about 45 nucleobases, about 25 to about 50 nucleobases, about 10 to about 30 nucleobases, about 14 to about 30 nucleobases, about 16 to about 24 nucleobases, about 18 to about 22 nucleobases, about 19 to about 21 nucleobases, or even about 18 to about 20 nucleobases in length. In some embodiments oligonucleotide is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60 nucleobases in length.

In some embodiments, the oligonucleotide targets a HBV sequence, and is complementary to a portion of the nucleic acid sequence of a HBV genome, e.g. as exemplified in Examples 1-9 and 11. In yet other embodiments, however, the oligonucleotide is not complementary to a portion of the nucleic acid sequence of a HBV genome, and as the inventors have shown here, e.g. as exemplified in Example 10, need not be complementary to achieve a desired prophylactic benefit, and/or the desired therapeutic benefit.

In some embodiments, the oligonucleotide targets a HBV sequence, and is complementary to a portion of the nucleic acid sequence of a HBV genome. An oligonucleotide and a target nucleic acid are complementary to each other when a sufficient number of nucleobases of the oligonucleotide can hydrogen bond with the corresponding nucleobases of the target nucleic acid. As contemplated herein, an oligonucleotide may not be complementary to the entire stretch of the target nucleic acid, and there may be areas that are not complementary. In some embodiments, the oligonucleotide is at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary to one or more portions of the nucleic acid sequence of a HBV genome—the % complementarity is determined over the entire length of the subject oligonucleotide, and the term “complementary” as used herein covers all of the recited ranges.

The oligonucleotides of the disclosure may exhibit complementarity to a 5′ UTR, a coding region, a 3′ UTR, an intron, an exon, or an exon/intron junction of a HBV genome. Target segments containing a start codon or a stop codon are also suitable target segments. A suitable target segment may specifically exclude a certain structurally defined region such as the start codon or stop codon. Exemplary HBV genomes include HBV genotypes (GT) A, B, C, D, E, F, G, H, I, and J, which are denoted as GT-A, GT-B, GT-C, GT-D, GT-E, GT-F, GT-G, GT-H, GT-I, and GT-J, respectively, or denoted as GTA, GTB, GTC, GTD, GTE, GTF, GTG, GTH, GTI, and GTJ, respectively. Exemplary HBV genomic nucleic acid sequences include, but are not limited to, those shown in Table 1.

TABLE 1
Exemplary HBV Genome sequences

| HBV Genotype | Accession No. | SEQ ID NO: |
|---|---|---|
| Consensus GTD | U95551.1 | SEQ ID NO: 3 |
| (HepG2.2.15 cells) | | |
| GTA | AJ309369.1 | SEQ ID NO: 667 |
| GTB | D00330.1 | SEQ ID NO: 668 |
| GTC | AB033550.1 | SEQ ID NO: 669 |
| GTD | KX470733.1 | SEQ ID NO: 670 |
| GTE | KX186584 | SEQ ID NO: 671 |
| GTF | KP718112 | SEQ ID NO: 672 |
| GTG | KX264500 | SEQ ID NO: 673 |
| GTH | KX264501 | SEQ ID NO: 674 |
| GTI | AF241411.1 | |
| GTJ | AB486012.1 | |

In some embodiments, the HBV nucleic acid is the sequence set forth in GENBANK Accession No. U95551.1 (provided herein as SEQ ID NO: 3). In some embodiments, the oligonucleotides target a sequence recited in SEQ ID NO: 3 or a portion thereof. In some embodiments, the oligonucleotides target a sequence at positions 1583-1602 of SEQ ID NO: 3.

Exemplary HBV nucleic acid target sequences include but are not limited to those shown in Table 2.

TABLE 2
Exemplary HBV Target Sequences

| HBV Target Sequence | SEQ ID NO: |
|---|---|
| CTTGG TCATG GGCCA TCAG | 1 |
| GCACT TCGCT TCACC TCTGC | 4 |
| TTTGTTTACGTCCCGTCGGC | 675 |
| TTTACGTCCCGTCGGCGCTGAATCCTGCGG | 676 |
| TTTACGTCCCGTCGGCGCTG | 677 |
| TTTACGCGGACTCCCCGTCT | 678 |
| TTGTTTACGTCCCGTCGGCG | 679 |
| TTGTTGACAAGAATCCTCAC | 680 |
| TTGAGAGAAGTCCACCACG | 681 |
| TTCTCCTGGCTCAGTTTACTAGTGCCATTT | 682 |
| TTCTCCTGGCTCAGTTTACT | 683 |
| TTACGTCCCGTCGGCGCTGA | 684 |
| TGTTTACGTCCCGTCGGCGC | 685 |
| TGTGCCTTCTCATCTGCCGG | 686 |
| TGTGCACTTCGCTTCACCTC | 687 |
| TGGTGGACTTCTCTCAATTT | 688 |
| TGGCTCAGTTTACTAGTGCC | 689 |
| TGGACTTCTCTCAATTTTCTAGGGG | 690 |
| TGGACTTCTCTCAATTTTCT | 691 |
| TGCCTTCTCATCTGCCGGAC | 692 |
| TGCCGGACCGTGTGCACTTC | 693 |
| TGCCGATCCATACTGCGGAA | 694 |
| TGCACTTCGCTTCACCTCTG | 695 |
| TGACAAGAATCCTCACAATA | 696 |
| TCTTTACGCGGACTCCCCGT | 697 |
| TCTTGTTGACAAGAATCCTC | 698 |
| TCTGTGCCTTCTCATCTGCC | 699 |
| TCTGCCGATCCATACTGCGG | 700 |
| TCTCTTTACGCGGACTCCCC | 701 |
| TCTCATCTGCCGGACCGTGT | 702 |
| TCGGCGCTGAATCCTGCGGA | 703 |
| TCCTGGCTCAGTTTACTAGT | 704 |
| TCCTCACAATACCGCAGAGT | 705 |
| TCCCGTCGGCGCTGAATCCT | 706 |
| TCCATACTGCGGAACTCCTA | 707 |
| TCATCTGCCGGACCGTGTGC | 708 |
| TCATCTCGAACGTTCACAGTCA | 709 |
| TCAGTTTACTAGTGCCATTT | 710 |
| TATTGTGAGGATTCTTGTCA | 711 |
| TAGGAGGCTGTAGGCATAAATTGGTCTGCG | 712 |
| TACGTCCCGTCGGCGCTGAA | 713 |
| TACGCGGACTCCCCGTCTGT | 714 |
| GTTTACGTCCCGTCGGCGCT | 715 |
| GTTGGTGCACTTCGCTTCAC | 716 |
| GTTGACAAGAATCCTCACAA | 717 |
| GTTCCCAGTATGGATCGGC | 718 |
| GTGTGCACTTCGCTTCACCT | 719 |
| GTGGTGGACTTCTCTCAATT | 720 |
| GTGGACTTCTCTCAATTTTCT | 721 |
| GTGGACTTCTCTCAATTTTC | 722 |
| GTGCCTTCTCATCTGCCGGA | 723 |
| GTGCACTTCGCTTCACCTCT | 724 |
| GTGAAGCGAAGTGCACACGG | 725 |
| GTCTGTGCCTTCTCATCTGC | 726 |
| GTCGGCGCTGAATCCTGCGG | 727 |
| GTCCCGTCGGCGCTGAATCC | 728 |
| GTATTGTGAGGATTCTTGTC | 729 |
| GGTGGACTTCTCTCAATTTT | 730 |
| GGGCGCACCTCTCTTTACGC | 731 |
| GGCTGTAGGCATAAATTGGT | 732 |
| GGCGCTGAATCCTGCGGACG | 733 |
| GGAGGAACCTTCAGGGAAGG | 734 |
| GGACTCCCCGTCTGTGCCTT | 735 |
| GGACCGTGTGCACTTCGCTT | 736 |
| GCTGTAGGCATAAATTGGT | 737 |
| GCTGAATCCTGCGGACGACC | 738 |
| GCTCAGTTTACTAGTGCCAT | 739 |
| GCGGACTCCCCGTCTGTGCC | 740 |
| GCGCTGAATCCTGCGGACGA | 741 |
| GCGCACCTCTCTTTACGCGG | 742 |
| GCCTTCTCATCTGCCGGACC | 743 |
| GCCGGACCGTGTGCACTTCG | 744 |
| GCCGATCCATACTGCGGAAC | 745 |
| GCCGATCCATACTGCGGAA | 746 |
| GCACTTCGCTTCACCTCTGC | 747 |
| GCACTAGTAAACTGAGCCAG | 748 |
| GCACCTCTCTTTACGCGGAC | 749 |
| GATCCATACTGCGGAACTCC | 750 |
| GACTCCCCGTCTGTGCCTTC | 751 |
| GACCGTGTGCACTTCGCTTC | 752 |
| GACAAGAATCCTCACAATAC | 753 |
| GAATCCTCACAATACCGCAG | 754 |
| GAAAATTGAGAGAAGTCCAC | 755 |
| CTTTGTTTACGTCCCGTCGG | 756 |
| CTTGTTGACAAGAATCCTCACAATACCGCA | 757 |
| CTTCTCATCTGCCGGACCGT | 758 |
| CTGTGCCTTCTCATCTGCCG | 759 |
| CTGGCTCAGTTTACTAGTGC | 760 |
| CTGGCTCAGTTTACTAGTG | 761 |
| CTGCCGGACCGTGTGCACTT | 762 |
| CTGCCGATCCATACTGCGGA | 763 |
| CTCTGCCGATCCATACTGCG | 764 |
| CTCCTGGCTCAGTTTACTAG | 765 |
| CTCCCCGTCTGTGCCTTCTC | 766 |
| CTCACAATACCGCAGAGTCT | 767 |
| CTATCAAGGTATGTTGCCCGTTTGTCCTCT | 768 |
| CGTGTGCACTTCGCTTCACC | 769 |
| CGTGGTGGACTTCTCTCAATTTTCTAGGGG | 770 |
| CGTGGTGGACTTCTCTCAATTTTCT | 771 |
| CGTGGTGGACTTCTCTCAAT | 772 |
| CGTGGTGGACTTCTCTCAAT | 773 |
| CGTCGGCGCTGAATCCTGCG | 774 |
| CGTCCCGTCGGCGCTGAATC | 775 |
| CGGGGCGCACCTCTCTTTAC | 776 |
| CGGCGCTGAATCCTGCGGAC | 777 |
| CGGACTCCCCGTCTGTGCCT | 778 |
| CGGACCGTGTGCACTTCGCT | 779 |
| CGCTGAATCCTGCGGACGAC | 780 |
| CGCGGACTCCCCGTCTGTGC | 781 |
| CGCACCTCTCTTTACGCGGA | 782 |
| CCTTTGTTTACGTCCCGTCG | 783 |
| CCTTCTCATCTGCCGGACCG | 784 |
| CCTTAGGCACTCCTGCCTCT | 785 |
| CCTGGCTCAGTTTACTAGTG | 786 |
| CCTCTGCCGATCCATACTGCGGAACTCCTA | 787 |
| CCTCTGCCGATCCATACTGC | 788 |
| CCTCTCTTTACGCGGACTCC | 789 |
| CCTCACAATACCGCAGAGTC | 790 |
| CCGTGTGCACTTCGCTTCAC | 791 |
| CCGTGTGCACTTCGCTGTTG | 792 |
| CCGTGTGCACTTCGCT | 793 |
| CCGTCTGTGCCTTCTCATCT | 794 |
| CCGTCGGCGCTGAATCCTGC | 795 |
| CCGGTCCGTGTGCACTTCGC | 796 |
| CCGGACCGTGTGCACTTCGC | 797 |
| CCGATCCATACTGCGGAACT | 798 |
| CCCGTCGGCGCTGAATCCTG | 799 |
| CCCCGTCTGTGCCTTCTCAT | 800 |
| CAGGTCCCCTAGAAGAAGAA | 801 |
| CACGGGGCGCACCTCTCTTT | 802 |
| CACCTCTCTTTACGCGGACT | 803 |
| CAAGAATCCTCACAATACCG | 804 |
| ATCTGCCGGACCGTGTGCAC | 805 |
| ATCCTCACAATACCGCAGAG | 806 |
| AGACTCGTGGTGGACTTCTCTCAATTTTCT | 807 |
| AGAATCCTCACAATACCGCA | 808 |
| ACGTCCCGTCGGCGCTGAAT | 809 |
| ACGCGGACTCCCCGTCTGTG | 810 |
| ACCTCTCTTTACGCGGACTC | 811 |
| ACCGTGTGCACTTCGCTTCA | 812 |
| ACCAATTTATGCCTACAGCG | 813 |
| ACAAGAATCCTCACAATACC | 814 |
| AATCCTCACAATACCGCAGA | 815 |
| AAGGTATGTTGCCCGTTTGT | 816 |
| AAGAATCCTCACAATACCGC | 817 |
| CTAGACTCGTGGTGGACTTC | 819 |
| CCTGCTGCTATGCCTCATCT | 820 |
| CTGCTGCTATGCCTCATCTT | 821 |
| TGCTGCTATGCCTCATCTTC | 822 |
| GCTGCTATGCCTCATCTTCT | 823 |
| CTGCTATGCCTCATCTTCTT | 824 |
| CCTATGGGAGTGGGCCTCAG | 825 |
| GCCATTTGTTCAGTGGTTCG | 826 |
| GGAGGCTGTAGGCATAAATT | 827 |
| GAGGCTGTAGGCATAAATTG | 828 |
| AGGCTGTAGGCATAAATTGG | 829 |
| GGCTGTAGGCATAAATTGGT | 830 |
| TTCAAGCCTCCAAGCTGTGC | 831 |
| TCAAGCCTCCAAGCTGTGCC | 832 |
| CAAGCCTCCAAGCTGTGCCT | 833 |
| AAGCCTCCAAGCTGTGCCTT | 834 |
| AGCCTCCAAGCTGTGCCTTG | 835 |
| GCCTCCAAGCTGTGCCTTGG | 836 |
| GAACTCCCTCGCCTCGCAGA | 837 |
| CACCATATTCTTGGGAACA | 838 |

In some embodiments, the HBV target comprises a sequence of SEQ ID NO: 1, 4, 675-838. In some embodiments, the HBV target comprises a sequence of SEQ ID NO: 1. In some embodiments, the HBV target comprises a sequence of SEQ ID NO: 4.

In some embodiments, the HBV target comprises the sequence recited in SEQ ID NO: 1 or a portion thereof or a variant thereof. In some embodiments, the oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary SEQ ID NO: 1.

In some embodiments, the HBV target comprises the sequence recited in SEQ ID NO: 4 or a portion thereof or a variant thereof. In some embodiments, the oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary SEQ ID NO: 4.

In some embodiments, the HBV target comprises positions 1583-1602 of SEQ ID NO: 3, or a portion thereof or a variant thereof. In some embodiments, the oligonucleotide is at least 30% at least 35%, at least 40%, at least 45%, at least 50%, at least 55%, at least 60%, at least 65%, at least 70%, at least 75%, at least 80%, at least 85%, at least 90%, at least 95%, at least 99%, or 100% complementary to positions 1583-1602 of SEQ ID NO: 3.

In some embodiments, the oligonucleotide has a nucleobase sequence of GCAGA GGTGA AGCGA AGTGC (SEQ ID NO: 2). A chart showing the nucleobase positions of SEQ ID NO: 2 is shown below:

| 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|
| G | C | A | G | A | G | G | T | G | A | A | G | C | G | A | A | G | T | G | C |

In some embodiments, the modified oligonucleotide of 20 linked nucleosides has a nucleobase sequence of GTGAA GCGAA GTGCA CACGG (SEQ ID NO: 5). A chart showing the nucleobase positions of SEQ ID NO: 5 is shown below:

| 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|
| G | T | G | A | A | G | C | G | A | A | G | T | G | C | A | C | A | C | G | G |

The oligonucleotides of the disclosure may comprise linked nucleosides, e.g. linked deoxynucleosides, linked ribonucleosides, and linked deoxyribonucleosides. Accordingly, the oligonucleotides of the disclosure may comprise deoxyribonucleotides, ribonucleotides, or a mixture of both. In some embodiments, the oligonucleotide is a modified oligonucleotide, e.g. the oligonucleotide comprises one or more sugar modifications, one or more modified internucleoside linkages, and/or one or more base modifications. Features of modified oligonucleotides of the disclosure are discussed in more detail below.

In some embodiments, the oligonucleotide is conjugated to a moiety or a conjugate, referred to as conjugated oligonucleotides. Features of conjugated oligonucleotides of the disclosure are discussed in more detail below. Conjugated oligonucleotides may be modified, or unmodified.

In some embodiments, an oligonucleotide of the disclosure comprises the sequence of any one of SEQ ID NOS: 10 to 666, or an oligonucleotide comprising 1, 2, 3, 4, 5, 6, 7, 8, 9, or 10 sequence modifications thereto. The term “sequence modification” as used herein refers to the identity of the sequence (e.g. a modification from an A to a T).

In some embodiments, an oligonucleotide of the disclosure comprises the sequence of any one of SEQ ID NOS: 10 to 666, or an oligonucleotide comprising at least 60%, 65%, 70%, 75%, 80%, 85%, 90%, 95% or 99% sequence identity thereto.

In some embodiments the vaccine comprises only one unique oligonucleotide—for clarity, the vaccine may comprise copies of the same unique oligonucleotide. In some embodiments the vaccine comprises a plurality of different oligonucleotides, e.g. at least 2, at least 3, at least 4, at least 5, at least 6, at least 7, at least 8, at least 9, or at least 10 or more different oligonucleotides of the disclosure.

b. Features of Modified Oligonucleotides

In some embodiments, the oligonucleotide is a modified oligonucleotide. As contemplated herein, modifications to oligonucleotides encompass substitutions or changes to internucleoside linkages, sugar moieties, and nucleobases. Modified oligonucleotides are often confer desirable properties such as, for example, enhanced cellular uptake, enhanced affinity for a nucleic acid target, increased stability in the presence of nucleases, or increased inhibitory activity. Chemically modified nucleosides may also be employed to increase the binding affinity for its target nucleic acid.

In some embodiments, a modified oligonucleotide of the disclosure comprises one or more modified internucleoside linkages. In some embodiments, a modified oligonucleotide of the disclosure comprises one or more modified internucleoside linkages, wherein the modified internucleoside linkage is a phosphorothioate internucleoside linkage. In some embodiments, the phosphorothioate modification comprises a mixture of Sp and Rp stereoisomers. In some embodiments, the phosphorothioate modification comprises a Sp stereoisomer. In some embodiments, the phosphorothioate modification comprises a Rp stereoisomer.

In some embodiments, a modified oligonucleotide of the disclosure comprises one or more sugar modifications. In some embodiments, a modified oligonucleotide of the disclosure comprises one or more linked nucleosides comprising a 2′ sugar modification (sugar modifications at the C2′ position). In exemplary embodiments, a modified oligonucleotide of the disclosure comprises one or more modifications to a 2′-deoxynucleoside, e.g. comprises one or more nucleosides comprising a 2′-O-methyl modification, 2′-O-(2-methoxyethyl) modification, 2′-fluoro modification, 2′-amino modification, 2′-O-propyl modification, 2′-O-butyl modification, 2′-O-cyclopropylmethyl modification, 2′-O-(2-hydroxyethyl) modification, 2′-O-[2-(methylamino)-2-oxyethyl] modification, 2′-O-2-[2-(N,N-dimethylamino) ethoxy]ethyl] modification, 2′-O-(2-dimethylaminoethyl) modification, 2′-O-(3-aminopropyl) modification, 2′-O-(2-aminopropyl) modification, 2′-O-(2-hydroxyisopropyl) modification, 2′-O-(2-methoxyisopropyl) modification, 2′-O-(2-dimethylaminoisopropyl) modification, 2′-O-(2-aminobutyl) modification, 2′-O-(2-hydroxybutyl) modification, 2′-O-(2-methoxybutyl) modification, 2′-O-(2-dimethylaminobutyl) modification, 2′-O-(2-aminocyclopropylmethyl) modification, 2′-O-(2-hydroxycyclopropylmethyl) modification, 2′-O-( 2-methoxycyclopropylmethyl) modification, 2′-O-(2-dimethylaminocyclopropylmethyl) modification, 2′-O-[(2-guanidinium)ethyl] modification, and/or a 2′-fluoro-arabino (2′-fluoro-ANA) modification.

In some embodiments, a modified oligonucleotide comprises one or more bicyclic sugar modified nucleosides. In some embodiments, a modified oligonucleotide of the disclosure comprises one or more bridged nucleic acids wherein the 2′-oxygen is linked through bridging carbons to the 4′ carbon of the ribose to form a bridged nucleic acid (BNA), e.g. linked through a 2′,4′-methylene bridge to form a locked nucleic acid (LNA), or e.g. in 2′,4′-constrained 2′-O-ethyl modifications ((R)-cET or(S)-cET), or e.g. in 2′,4′-constrained 2′-O-methoxyethyl modifications ((R)-cMOE or(S)-cMOE).

In some embodiments, a modified oligonucleotide comprises one or more carbocyclic LNA (cLNA) where the 2′-oxygen atom in LNA is replaced with a carbon atom. In some exemplary embodiments, a modified oligonucleotide of the disclosure comprises one or more methanocarba bicyclo[3.1.0]hexane sugar either in the North C2′-exo (N-MC) or South C3′-exo forms (S-MC), or 2′-F-NMC. In some embodiments, a modified oligonucleotide comprises (R)-Me-cLNA, or(S)-Me-cLNA, or F-LNA, or methylene-cLNA as shown below.

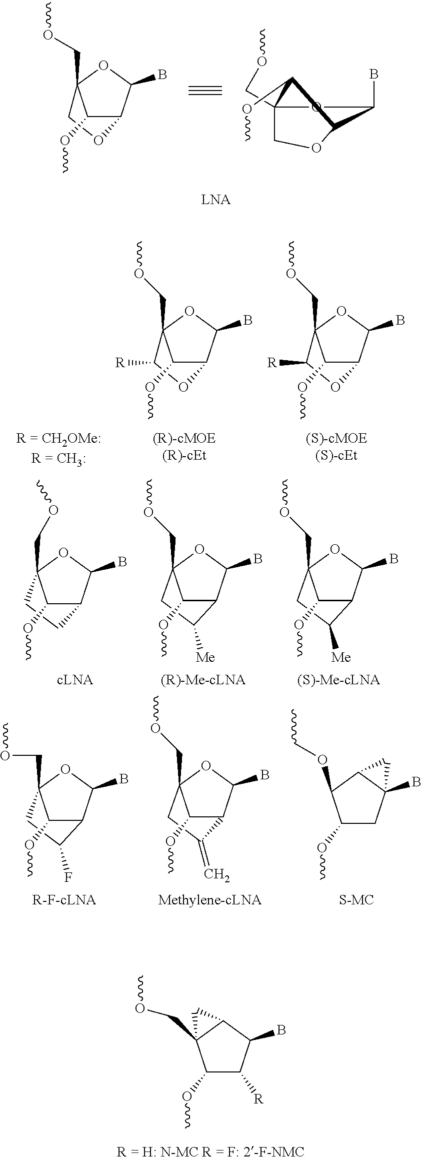

In some embodiments, a modified oligonucleotide comprises one or more of an «-L-LNA, bcDNA, or tcDNA as shown below.

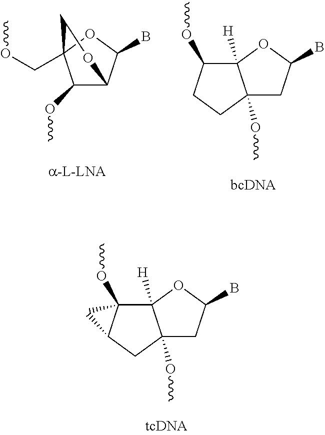

In some embodiments, a modified oligonucleotide of the disclosure comprises one or more morpholino modifications wherein a morpholino group is in place of the ribose sugar. In some embodiments, a modified oligonucleotide of the disclosure comprises phosphorodiamidate morpholino modification wherein the sugar-phosphate backbone is replaced with a phosphorodiamidate morpholino moiety. In some embodiments, a modified oligonucleotide of the disclosure comprises one or more peptide nucleic acid (PNA) modification wherein the sugar-phosphate backbone or sugar-phosphorothioate backbone is replaced with a peptide backbone. In some embodiments, a modified oligonucleotide of the disclosure comprises one or more glycol nucleic acid (GNA) modification, which sometimes is also referred to as glycerol nucleic acid, wherein a propylene glycol group is in place of the ribose sugar. In some embodiments, a modified oligonucleotide of the disclosure comprises one or more phosphate analog E vinylphosphonate (E-VP), and the amide backbone linkage, as shown below.

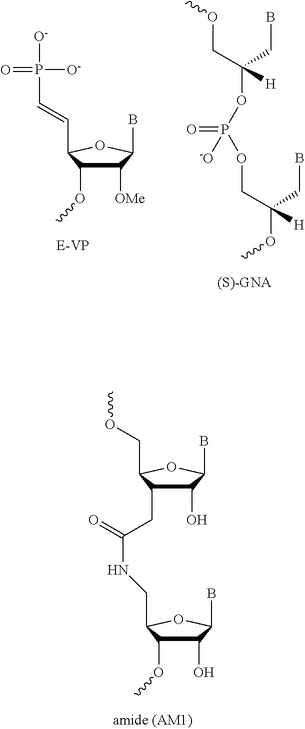

In some embodiments, a modified oligonucleotide of the disclosure comprises one or more base modifications. In some embodiments, a modified oligonucleotide of the disclosure comprises one or more nucleosides that are a 5-methylcytidine. In some embodiments, a modified oligonucleotide of the disclosure comprises one or more nucleosides that are a N1-Methyl-pseudouridine.

In some embodiments, a modified oligonucleotide of the disclosure comprises one or more modifications to the 3′-end and/or 5′-end of the oligonucleotides, e.g. 3′- and/or 5′-amino modifications.

In some embodiments, a modified oligonucleotide of the disclosure comprises segmented oligonucleotides, creating one or more internal regions (gaps) having a plurality of nucleotides that supports RNaseH cleavage, which are positioned between external regions (5′ and 3′ wing segments) having a plurality of nucleotides that are chemically distinct from the nucleosides of the internal regions. In the case of an oligonucleotide exhibiting complementarity to a target having a segmented motif, the gap segment can hybridize with a target RNA sequence for endonuclease cleavage, while the wing segments comprise modified nucleosides. In some embodiments, the regions of a gaps are differentiated by the types of sugar moieties comprising each distinct region. The types of sugar moieties that are used to differentiate the regions of a gap may, in some embodiments, include β-D-ribonucleosides, β-D-deoxyribonucleosides, 2′-modified nucleosides (such 2′-modified nucleosides may include 2′-MOE and 2′-O—CH₃, among others as described in the preceding paragraphs, and bicyclic sugar modified nucleosides (such bicyclic sugar modified nucleosides may include those having a constrained ethyl). In some embodiments, nucleosides in the wings may include modified sugar moieties, including, for example 2′-MOE and bicyclic sugar moieties such as constrained ethyl or LNA. In some embodiments, the wings may include several modified and unmodified sugar moieties as described in the preceding paragraphs. In some embodiments, wings may include various combinations of 2′-MOE nucleosides, bicyclic sugar moieties such as constrained ethyl nucleosides or LNA nucleosides, and 2′-deoxynucleosides. There can be a single internal region (gap), or can be further segmented with separator segments, thus resulting in a discontinuous gap segments, which, in some embodiments provides improved activity over conventional oligonucleotides that have a continuous gap (e.g., reduction of HBsAg levels, HBeAg levels, and/or HBcAg levels in the serum or other body fluid or tissue). One or more separator segments placed directly in between gap segments can be beneficial. Examples 1-11 show the advantages of the oligonucleotides that comprise a variety of segmented oligonucleotides.

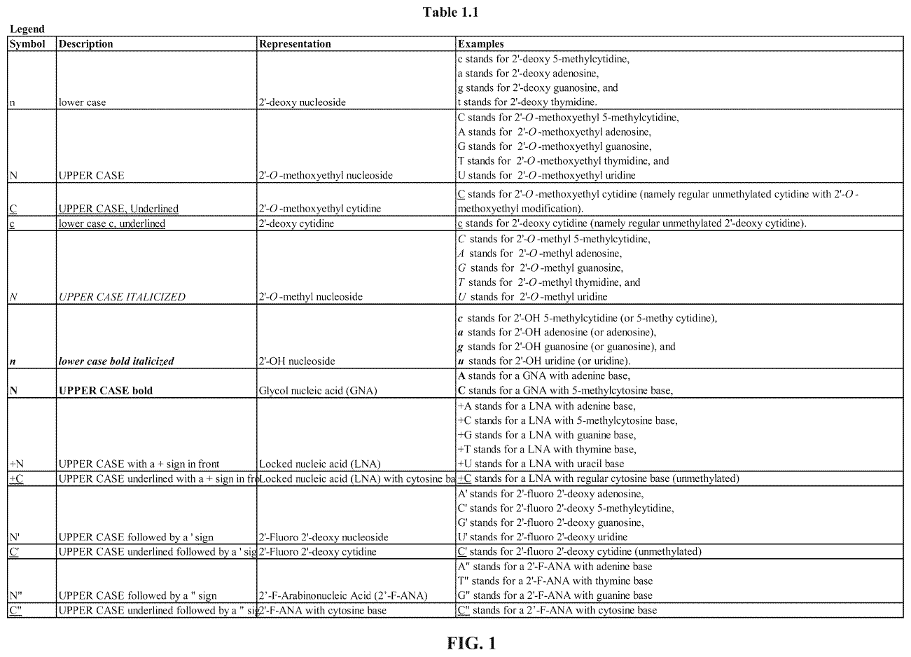

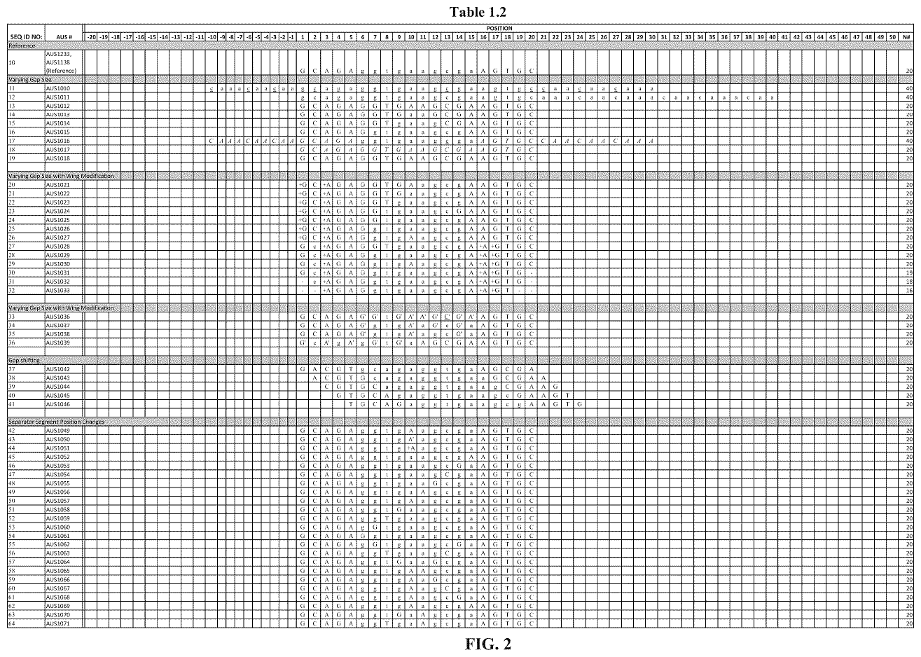

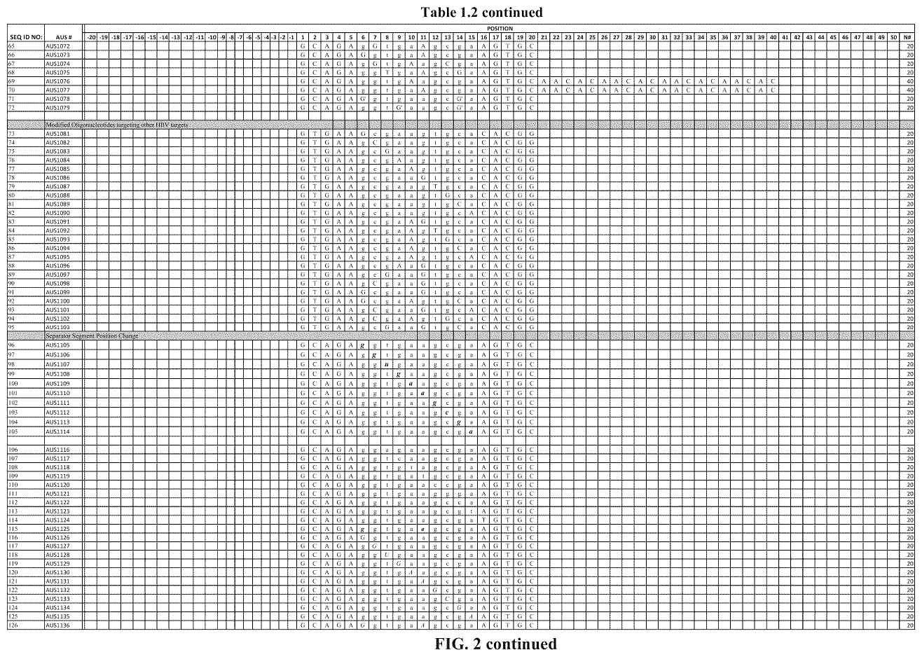

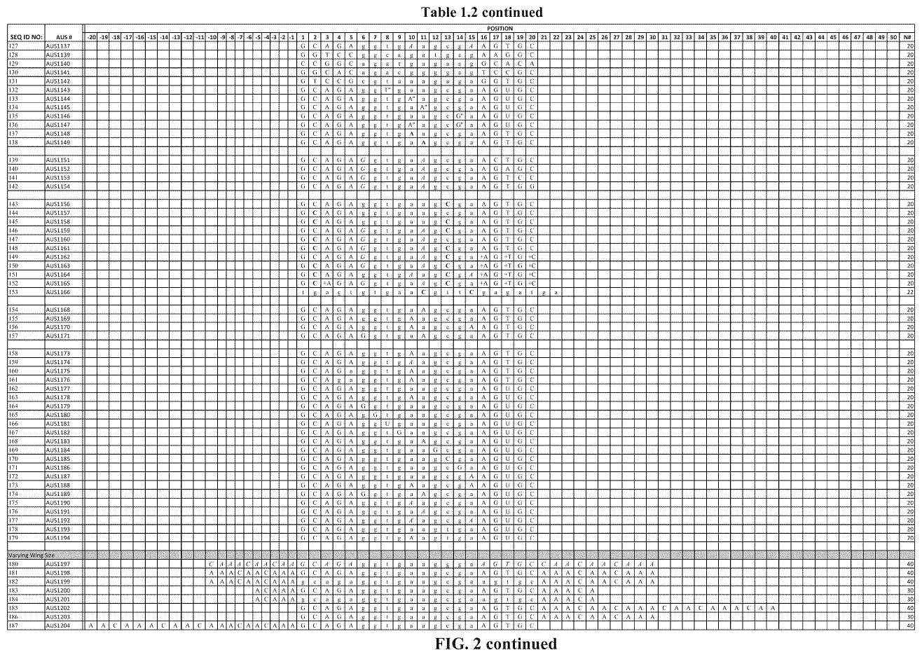

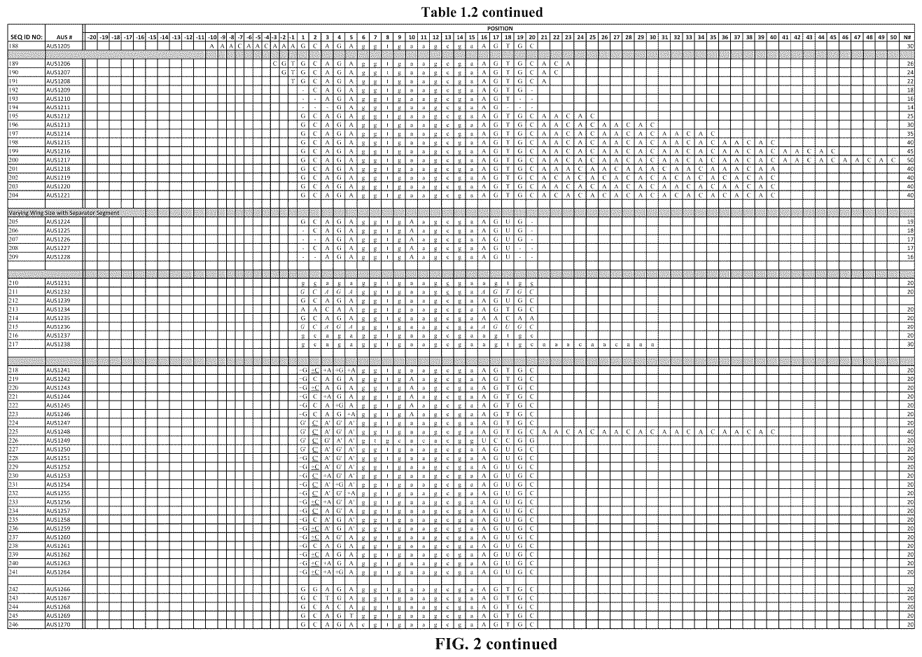

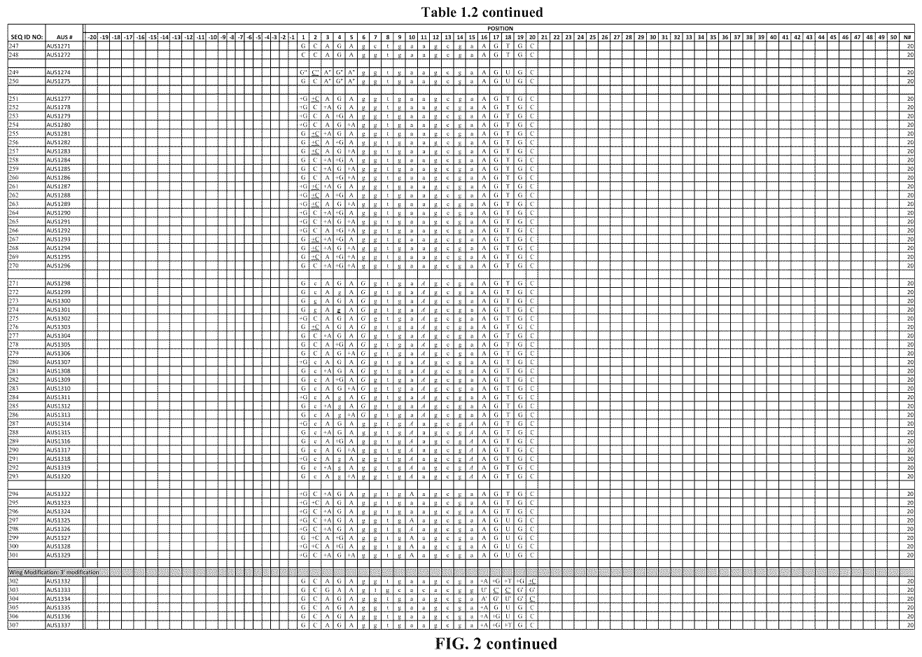

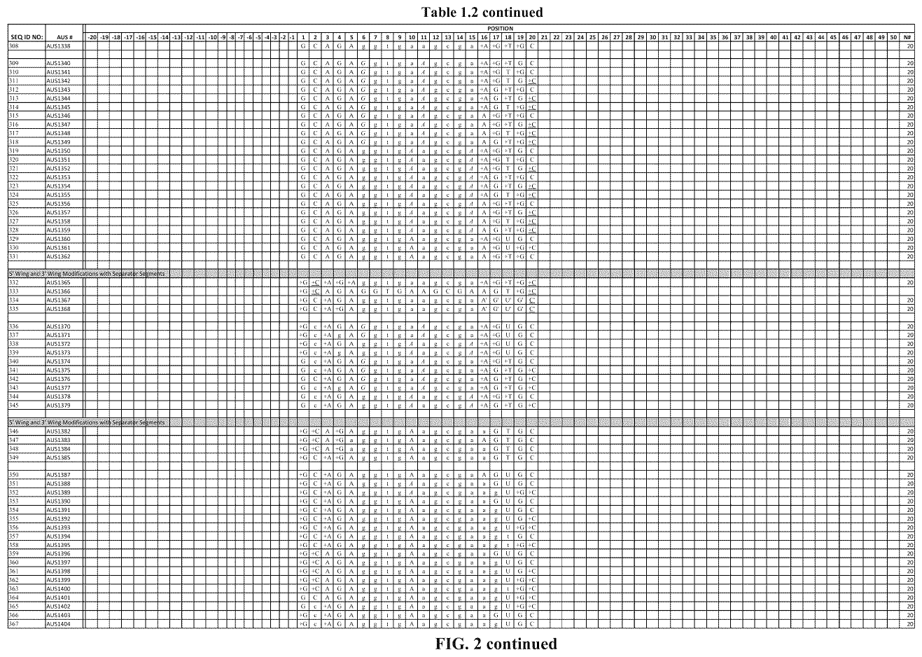

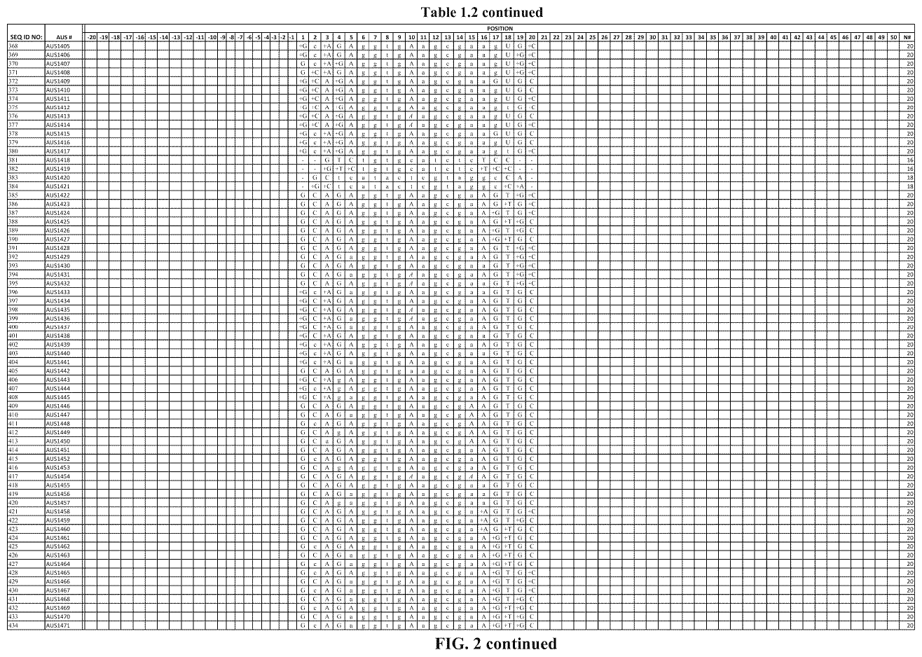

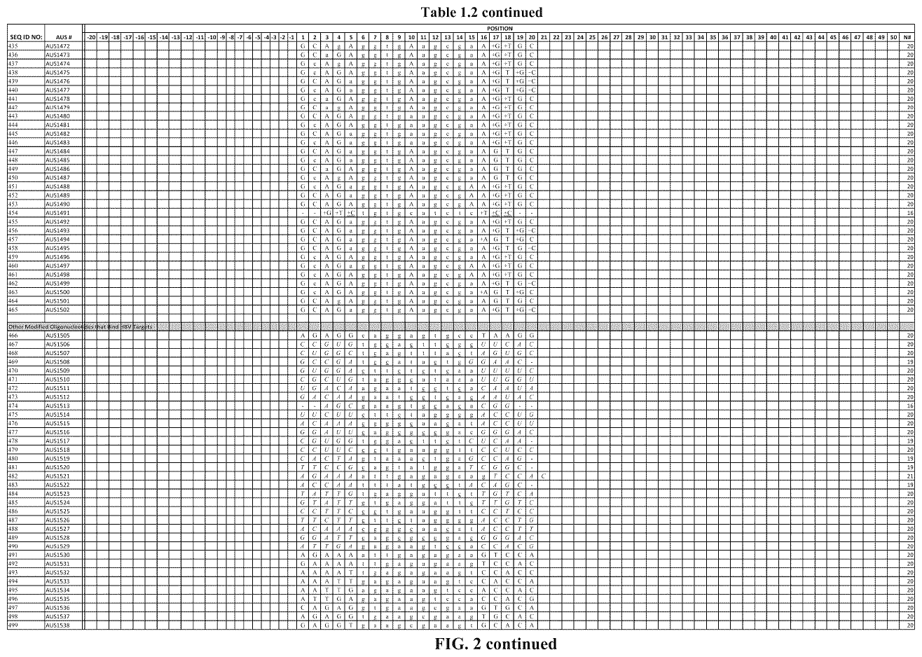

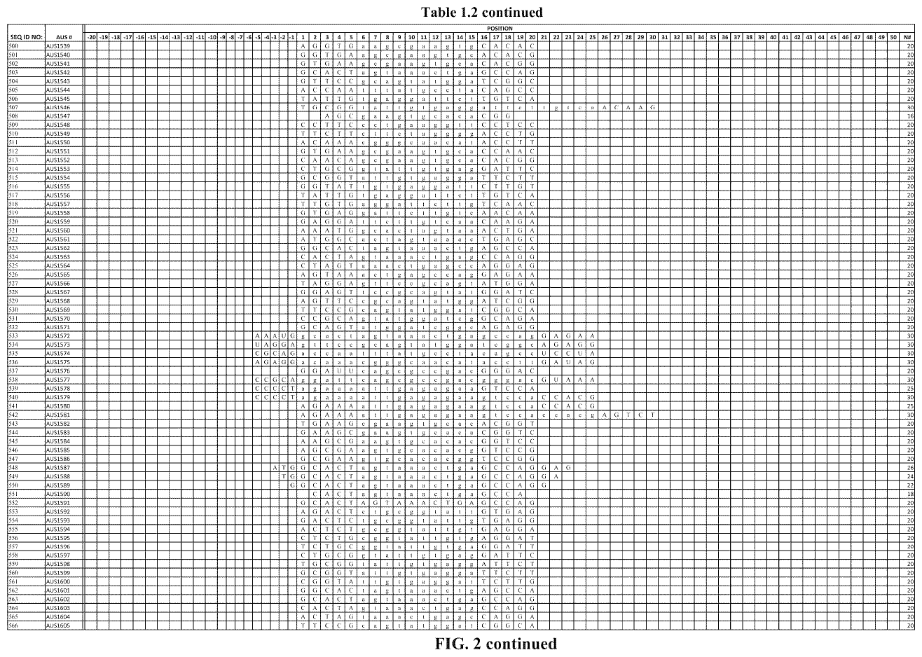

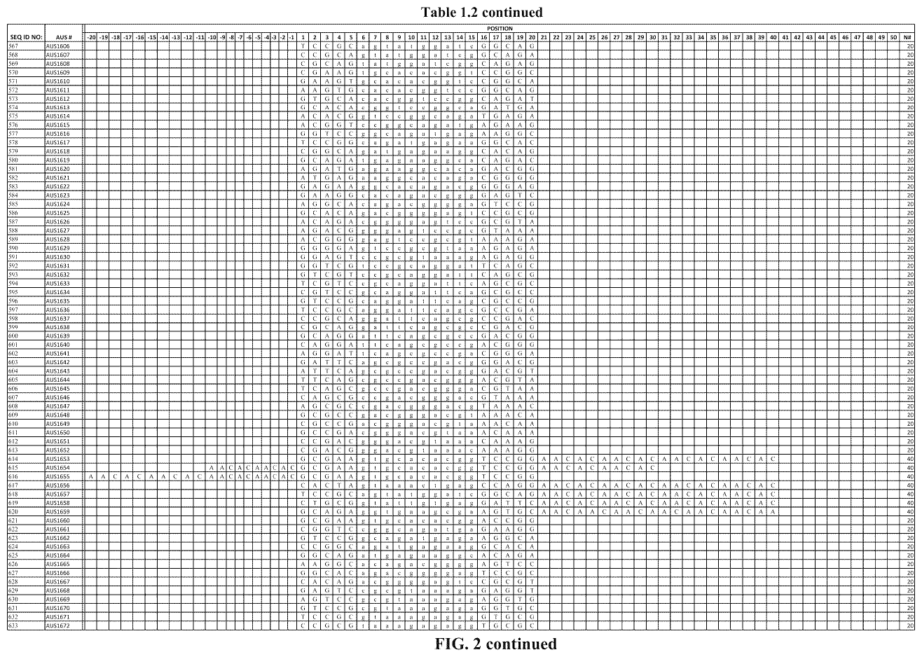

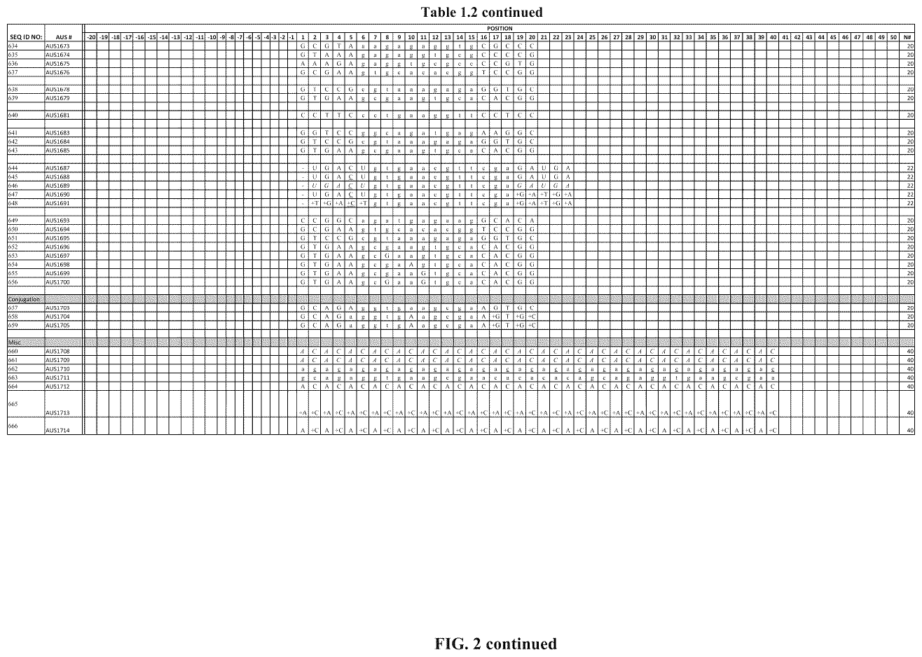

Exemplary modifications of the disclosure include, but are not limited, to those listed in Table 1.1 (FIG. 1). Exemplary modified oligonucleotides of the disclosure include, but are not limited to, those exemplified in Table 1.2 (FIG. 2, as read in light of the legend of FIG. 1). In some embodiments, the modified oligonucleotide comprising the sequence of any one of AUS1010 to AUS1714.

c. Conjugated Oligonucleotides

The oligonucleotides of the disclosure may be further covalently linked to one or more conjugate groups/moieties. In some embodiments, such groups enhance the activity, cellular distribution and/or cellular uptake of the resulting vaccines. Exemplary conjugate groups include, but are not limited to, carbohydrate moieties (e.g, N-Acetylgalactosamine-containing carbohydrate chains), and lipid moieties (e.g., cholesterol and phospholipids). Other conjugate groups include, but are not limited to-phospholipids, biotin or other affinity tags (e.g., streptavidin), polymers (e.g., polyethylene glycol (PEG), polylysine), cell-penetrating peptides (e.g., Tat, Penetratin), enzymes (e.g., horseradish peroxidase, alkaline phosphatase), antibodies or antibody fragments (e.g., anti-HER2, anti-EGFR), proteins or peptides (e.g., growth factors, cytokines), metal nanoparticles (e.g., gold nanoparticles), small molecule drugs (e.g., doxorubicin, paclitaxel), phenazine, folate, phenanthridine, anthraquinone, acridine, fluoresceins, rhodamines, coumarins, and dyes.

The oligonucleotides of the disclosure may also be modified to have one or more stabilizing groups that are generally attached to one or both termini of oligonucleotides to enhance properties such as, for example, nuclease stability. Included in stabilizing groups are cap structures. These terminal modifications can protect the oligonucleotide having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps.

Conjugated oligonucleotides of the disclosure may be modified as described in the preceding section.

d. Hepatitis B Viral Antigens

As provided herein, the vaccines of the disclosure comprise at least one oligonucleotide and at least one HBV antigen, the two components which can be administered in two separate compositions, or together in a single composition. In some embodiments, the vaccine comprises a mixture of different HBV antigens, e.g. at least 2, 3, 4, or 5 different HBV antigens.

In some embodiments, the HBV antigen comprises a HBV surface antigen (HBsAg), a HBV e antigen (HBeAg), and/or a HBV core antigen (HBcAg). In some embodiments, the HBV antigen is a human HBV antigen. In some embodiments, the HBV antigen comprises a Hepatitis B small surface antigen (Small-HBs (S-HBs)). In some embodiments, the HBV antigen comprises a Hepatitis B medium surface antigen (Middle-HBs (M-HBs)). In some embodiments, the HBV antigen comprises a Hepatitis B large surface antigen (Large-HBs (L-HBs)). In some embodiments, the HBV antigen comprises a mixture of Hepatitis B small and medium surface antigen. In some embodiments, the HBV antigen comprises a mixture of Hepatitis B small and large surface antigen. In some embodiments, the HBV antigen comprises a mixture of Hepatitis B medium and large surface antigen. In some embodiments, the HBV antigen comprises a mixture of Hepatitis B small, medium and large surface antigens. In some embodiments, the HBV antigen is from a HBV of GT-A, GT-B, GT-C, GT-D, GT-E, GT-F, GT-G, GT-H, GT-I, or GT-J genotype.

The HBV antigen can be isolated from patients, can be recombinantly produced (e.g. in mammalian cells, yeast cells, bacterial cells, insect cells), or alternatively, can be synthesized.

e. Adjuvants

As provided herein, the vaccines of the disclosure comprise at least one oligonucleotide and at least one HBV antigen, and can further comprise an adjuvant. If the vaccine is provided as a single composition, the composition can comprise an adjuvant. If the vaccine is provided in separate compositions, then either the oligonucleotide component or the antigen component can comprise, or both can comprise an adjuvant. Exemplary adjuvants include, but are not limited to, an aluminum hydroxide, a toll-like receptor 9 (TLR9) agonist adjuvant, or an oligonucleotide. An exemplary oligonucleotide adjuvant comprises CpG1018, a 22-mer CpG oligodeoxynucleotide containing sequence with a modified phosphorothioate backbone, 5′-tgactgtgaacgttcgagatga-3′ (SEQ ID NO: 839) (each lower case represents a 2′-deoxy nucleoside as presented in Table 1.1 of FIG. 1).

### II. Methods

a. Prophylactic Methods

In one aspect, the disclosure provides methods of preventing an HBV infection in a subject, comprising administering a prophylactically effective amount of at least one oligonucleotide described herein in conjunction with at least one HBV antigen (i.e. the vaccine), or pharmaceutical composition(s) comprising same. As has been noted throughout, the oligonucleotide and the HBV antigen can be present together in a single composition, or provided separately for such prophylactic use.

In some embodiments, the subject is at risk for an HBV-related condition. This includes subjects having one or more risk factors for developing an HBV-related condition, including sexual exposure to a subject infected with Hepatitis B virus, living in the same house as an subject with a lifelong hepatitis B virus infection, exposure to human blood infected with the hepatitis B virus, injection of illicit drugs, being a person who has hemophilia, and visiting an area where hepatitis B is common.

b. Therapeutic Methods

In another aspect, the disclosure provides methods of treating a subject with an HBV infection, or with an HBV-related disease, disorder, or condition, the methods comprising administering a therapeutically effective amount of at least one oligonucleotide described herein in conjunction with at least one HBV antigen (i.e. the vaccine), or pharmaceutical composition(s) comprising same. As has been noted throughout, the oligonucleotide and the HBV antigen can be present together in a single composition, or provided separately for such prophylactic use.

In some embodiments, the subject is a human and the subject is suffering from a hepatitis B virus infection from a human hepatitis B virus. In exemplary embodiments, the human hepatitis B virus may be any of the human geographical genotypes: A (Northwest Europe, North America, Central America); B (Indonesia, China, Vietnam); C (East Asia, Korea, China, Japan, Polynesia, Vietnam); D (Mediterranean area, Middle East, India); E (Africa); F (Native Americans, Polynesia); G (United States, France); H (Central America), I (Asia including India and Vietnam), or J (Japan).

Examples of HBV-related diseases, disorders or conditions include, but are not limited to, chronic HBV infection, jaundice, liver cancer, liver inflammation, liver fibrosis, liver cirrhosis, liver failure, diffuse hepatocellular inflammatory disease, hemophagocytic syndrome, serum hepatitis, Hepatitis Delta virus (HDV) infection (e.g. in subject co-infected with HBV), Hepatitis C virus (HCV) infection (e.g. in subject co-infected with HBV), inflammation, fibrosis, and HBV viremia. HBV-related conditions or disorders can have symptoms which may include any or all of the following: flu-like illness, weakness, aches, headache, fever, loss of appetite, diarrhea, nausea and vomiting, pain over the liver area of the body, clay- or grey-colored stool, itching all over, and dark-colored urine, which when coupled with a positive test for presence of a hepatitis B virus, a hepatitis B viral antigen, or a positive test for the presence of an antibody specific for a hepatitis B viral antigen indicate an HBV-related condition or disorder.

In some embodiments, a positive indicator of treatment, includes decreased fatigue, decreased flu-like symptoms, increase in appetite, decreased nausea, decreased joint pain, decreased jaundice, decreased pain in the abdomen, decreased weakness, decreased weight loss, reduction in breast enlargement in men, decreased rash on the palms, decreased difficulty with blood clotting, decreased cirrhosis, decreased spider-like blood vessels on the skin, increased Vitamins A and D absorption, decreased tumor growth, decreased tumor volume, decreased headache, decreased fever, decreased diarrhea, decreased pain over the liver area of the body, decreased clay- or grey-colored stool, decreased itching, decreased dark-colored urine, and decreased nausea and vomiting can be indicative of inhibition of HBV expression.

In some embodiments, administration of a therapeutically effective amount of a vaccine of the disclosure is accompanied by monitoring of HBV mRNA levels in the serum or tissue (or other bodily sample) of a subject to determine a subject's response to administration of the vaccine. In some embodiments, administration of a therapeutically effective amount of a vaccine of the disclosure is accompanied by monitoring of HBV DNA levels in the serum or tissue (or other bodily sample) of a subject to determine a subject's response to administration of the vaccine. In some embodiments, administration of a therapeutically effective amount of a vaccine of the disclosure is accompanied by monitoring of HBV protein levels in the serum or tissue (or other bodily sample) of a subject to determine a subject's response to administration of the vaccine. In some embodiments, administration of a therapeutically effective amount of a vaccine of the disclosure is accompanied by monitoring of HBV S antigen (HBsAg) levels in the serum or tissue (or other bodily sample) of a subject to determine a subject's response to administration of the vaccine. In some embodiments, administration of a therapeutically effective amount of a vaccine of the disclosure is accompanied by monitoring of HBV E antigen (HBeAg) levels in the serum or tissue (or other bodily sample) of a subject to determine a subject's response to administration of the vaccine. A subject's response to administration of the vaccine is used by a physician to determine the amount and duration of therapeutic intervention.

In some embodiments, administration of a vaccine of the disclosure results in reduction of HBV expression by at least 30%, 50%, 65%, 90%, 97%, 99%, 99.7%, 99.9%, 99.97% or 99.99%, which respectively corresponds to approximately 0.15, 0.3, 0.5, 1, 1.5, 2, 2.5, 3, 3.5, or 4 Log 10 reduction, or a range defined by any two of these values.

In some embodiments, administration of a vaccine of the disclosure results in decreased symptoms associated with the HBV-related condition and decreased HBV-related markers in the blood. In some embodiments, administration of a vaccine of the disclosure decreases HBV RNA levels, HBV DNA levels, HBV protein levels, HBsAg levels, HBcAg levels, and/or HBeAg levels by at least about 30%, 50%, 60%, 68%, 80%, 90%, 97%, 99%, 99.7%, 99.9%, 99.97%, 99.99 or 99.997%, which respectively corresponds to approximately 0.15, 0.3, 0.4, 0.5, 0.7, 1, 1.5, 2, 2.5, 3, 3.5, 4, or 4.5 Log 10 reduction, or a range defined by any two of these values.

In some embodiments, the vaccines of the disclosure inhibit HBV mRNA expression in the subject. In some embodiments, the vaccines of the disclosure inhibit HBV DNA levels in the subject. In some embodiments, the vaccines of the disclosure inhibit HBV protein levels and/or antigen levels in the subject. The vaccines of the disclosure, when administered to a subject, can decrease the levels of HBV mRNA, DNA, and/or protein, including HBV antigens such as, but not limited to HBsAg and HBeAg. In some embodiments, the vaccines of the disclosure result in an increase in HBV antibody levels.

Certain embodiments provide a method of reducing HBV mRNA expression in a subject comprising administering to the subject an oligonucleotide/HBV antigen vaccine of the disclosure. In some embodiments, reduction of HBV mRNA expression in the subject prevents, ameliorates or treats an HBV-related disease, disorder or condition. In some embodiments, reduction of HBV mRNA expression in the subject ameliorates or treats HBV infection. In some embodiments, reduction of HBV mRNA expression in the subject prevents, ameliorates or treats liver disease. In some embodiments, administration of a vaccine of the disclosure decreases HBV RNA levels by at least about 30%, 50%, 60%, 68%, 80%, 90%, 97%, 99%, 99.7%, 99.9%, 99.97%, 99.99 or 99.997%, which respectively corresponds to approximately 0.15, 0.3, 0.4, 0.5, 0.7, 1, 1.5, 2, 2.5, 3, 3.5, 4, or 4.5 Log 10 reduction, or a range defined by any two of these values.

Certain embodiments provide a method of reducing HBV protein levels in a subject comprising administering to the subject an oligonucleotide/HBV antigen vaccine of the disclosure. In some embodiments, reduction of HBV protein levels in the subject prevents, ameliorates or treats an HBV-related disease, disorder or condition. In some embodiments, reduction of HBV protein levels in a subject ameliorates or treats HBV infection. In some embodiments, reduction of HBV protein levels in the subject prevents, ameliorates or treats liver disease. In some embodiments, administration of a vaccine of the disclosure decreases HBV protein levels by at least about 30%, 50%, 60%, 68%, 80%, 90%, 97%, 99%, 99.7%, 99.9%, 99.97%, 99.99 or 99.997%, which respectively corresponds to approximately 0.15, 0.3, 0.4, 0.5, 0.7, 1, 1.5, 2, 2.5, 3, 3.5, 4, or 4.5 Log 10 reduction, or a range defined by any two of these values.

Certain embodiments provide a method of reducing HBV DNA levels in a subject comprising administering to the subject an oligonucleotide/HBV antigen vaccine of the disclosure. In some embodiments, reduction of HBV DNA levels in the subject prevents, ameliorates or treats an HBV-related disease, disorder or condition. In some embodiments, administration of a vaccine of the disclosure decreases HBV DNA levels by at least about 30%, 50%, 60%, 68%, 80%, 90%, 97%, 99%, 99.7%, 99.9%, 99.97%, 99.99 or 99.997%, which respectively corresponds to approximately 0.15, 0.3, 0.4, 0.5, 0.7, 1, 1.5, 2, 2.5, 3, 3.5, 4, or 4.5 Log 10 reduction, or a range defined by any two of these values.

Certain embodiments provide a method of reducing one or more HBV antigen levels in a subject comprising administering to the subject an oligonucleotide/HBV antigen vaccine of the disclosure. In some embodiments, the antigen is HBsAg, HBcAg, and/or HBeAg. In some embodiments, reduction of HBV antigen levels in the subject prevents, ameliorates or treats an HBV-related disease, disorder or condition. In some embodiments, reduction of HBV antigen levels in the subject prevents, ameliorates or treats liver disease. In some embodiments, administration of a vaccine of the disclosure decreases HBV antigen levels by at least about 30%, 50%, 60%, 68%, 80%, 90%, 97%, 99%, 99.7%, 99.9%, 99.97%, 99.99 or 99.997%, which respectively corresponds to approximately 0.15, 0.3, 0.4, 0.5, 0.7, 1, 1.5, 2, 2.5, 3, 3.5, 4, or 4.5 Log 10 reduction, or a range defined by any two of these values. In some embodiments, administration of a vaccine of the disclosure decreases HBsAg levels by at least about 30%, 50%, 60%, 68%, 80%, 90%, 97%, 99%, 99.7%, 99.9%, 99.97%, 99.99 or 99.997%, which respectively corresponds to approximately 0.15, 0.3, 0.4, 0.5, 0.7, 1, 1.5, 2, 2.5, 3, 3.5, 4, or 4.5 Log 10 reduction, or a range defined by any two of these values.

Certain embodiments provide a method of increasing the level of circulating antibodies to HBV proteins and/or HBV antigens in a subject comprising administering to the subject an oligonucleotide/HBV antigen vaccine of the disclosure. In some embodiments, the antibody is to the HBsAg, HBcAg, and/or HBeAg. In some embodiments, increase of levels of antibody against HBV antigen in the subject prevents, ameliorates or treats an HBV-related disease, disorder or condition. In some embodiments, administration of a vaccine of the disclosure increases antibodies against HBV proteins and/or HBV antigens by at least about 1.25-fold, 1.5-fold, 2-fold, 5-fold, 10-fold, 20-fold, 50-fold, 100-fold, 250-fold, 500-fold, 1000-fold, 10000-fold, or even 1000000-fold, or a range defined by any two of these values.

Certain embodiments provide a method of increasing the T cell response to HBV antigens in a subject comprising administering to the subject an oligonucleotide/HBV antigen vaccine of the disclosure. In some embodiments, increase in T cell response in the subject prevents, ameliorates or treats an HBV-related disease, disorder or condition. In some embodiments, the increase in T cell response in the subject prevents in the subject prevents, ameliorates or treats liver disease. In some embodiments, the increase in T cell response in the subject is increased by at least about 1.25-fold, 1.5-fold, 2-fold, 5-fold, 10-fold, 20-fold, 50-fold, 100-fold, 250-fold, 500-fold, 1000-fold, 10000-fold, or even 1000000-fold, or a range defined by any two of these values.

In some embodiments, the amount of HBV antigen may be sufficiently decreased to result in seroconversion, defined as serum HBsAg undetectable if monitoring HBsAg as the determinant for seroconversion.

In some embodiments, the amount of an HBV antigen is undetectable for at least 1, 2, 3, 4, 5, 6, or more months following the treatment. In some embodiments, administration to the subject the oligonucleotide/HBV antigen vaccine of the disclosure results in a functional cure (HBsAg undetectable for six months or more). In some embodiments, administration to the subject the oligonucleotide/HBV antigen vaccine of the disclosure results in true cure (e.g. clearance of HBV DNA/mRNA and HBV antigens).

Certain embodiments provide a method for treating a subject with a HBV infection, HBV related disease, disorder or condition comprising: a) identifying said subject with the HBV infection, HBV related disease, disorder or condition, and b) administering to said subject a therapeutically effective amount of an oligonucleotide/HBV antigen vaccine of the disclosure. In some embodiments, the therapeutically effective amount of the oligonucleotide/HBV antigen vaccine of the disclosure administered to the subject treats or decreases the HBV infection, or HBV related disease, disorder or condition, or a symptom thereof, in the subject. In some embodiments, the HBV related disease, disorder or condition is a liver disease. In some embodiments, the related disease, disorder or condition is chronic HBV infection, jaundice, liver cancer such as hepatocellular carcinoma, liver inflammation, liver fibrosis, liver cirrhosis, liver failure, diffuse hepatocellular inflammatory disease, hemophagocytic syndrome, serum hepatitis, HBV viremia, liver disease-related to transplantation, or any combination thereof.

It is noted that reduction of levels or expression of a HBV nucleic acid upon treatment can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, CA and used according to manufacturer's instructions.

Likewise, it is noted that reduction of HBV antigen levels and/or increase in HBV antibody levels can be assessed using standard techniques. Protein levels of HBV and antibodies can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art.

c. Subjects

The subjects of the disclosure eligible for the prophylactic and therapeutic vaccines of the disclosure include all mammals, for example humans, and non-human primates. In some embodiments, the subject is a human. In some embodiments, the subject is a non-human primate, for example a cynomolgus monkey, rhesus monkey, or chimpanzee. In some embodiments, the subject is a rodent, such as a mouse or rat. Subjects can also include domestic pets (e.g. dogs, cats), and livestock/farm animals (e.g. cows, goats, sheep, pigs).

In some embodiments, the subject is human pediatric subject, >0 and <13 years of age.

In some embodiments, the subject is human adolescent subject, ≥13 and <18 years of age.

In some embodiments, the subject is human adult subject, ≥18 years of age.

In some embodiments, the subject is human adult geriatric subject, ≥65 years of age.

d. Dosing and Administration

The disclosure provides methods comprising administering an effective dose of the oligonucleotide/HBV antigen vaccine of the disclosure, or pharmaceutical composition comprising the same, to a subject in need thereof.

Dosing may need to be adjusted by a medical practitioner, based on, for example, the age of the subject. The vaccines of the disclosure are useful for pediatric, adolescent, adult, and geriatric adult subjects, as noted above.

A variety of routes are contemplated for administration, as noted here, including oral, pulmonary, rectal, parenteral, transdermal, subcutaneous, intravenous, intraarterial, intramuscular, intraperitoneal, inhalational, buccal, sublingual, intrapleural, intrathecal, intranasal, intracranial, intrathecal, intraocular, or intracerebroventricular administration. In exemplary embodiments, the route of administration is subcutaneous. In exemplary embodiments, the route of administration is intravenous. In exemplary embodiments, the route of administration is intradermal. In exemplary embodiments, the route of administration is intramuscular.

In some embodiments the oligonucleotide component and HBV component of the vaccine are administered as separate compositions, and are administered simultaneously, e.g. at two adjacent sites of administration. In some embodiments the oligonucleotide component and HBV component of the vaccine are administered as separate compositions, and are administered sequentially at two adjacent sites of administration. In some embodiments the oligonucleotide component and HBV component of the vaccine are administered as separate compositions, and are administered sequentially at two simultaneous sites of administration. The sequential administration may be at least about 1, 2, 3, 5, 10, 15, 20, 25, 30, 45, 60, 90, or even 120 minutes apart, or a range defined by any two of these values.

In some embodiments, the oligonucleotide and the HBV antigen vaccine are administered as separate compositions, and are administered sequentially, e.g., as exemplified in Example 11. In some embodiments, the oligonucleotide is administered before the HBV antigen. In some embodiments, multiple doses of the oligonucleotide are administered before the HBV antigen. In some embodiments, multiple doses of the oligonucleotide are administered before multiple doses of the HBV antigen. In some embodiments, the 1, 2, 3, 4, 5, or more doses of the oligonucleotide are administered before the 1, 2, 3, 4, 5 or more doses of the HBV antigen. In some embodiments, the 1, 2, 3, 4, or 5 doses of the oligonucleotide are administered before the 1, 2, 3, 4, or 5 doses of the HBV antigen. In exemplary embodiments, 4 doses of the oligonucleotide are administered before 3 doses of the HBV antigen. In exemplary embodiments, 4 doses of the oligonucleotide are administered before 4 doses of the HBV antigen. In exemplary embodiments, 3 doses of the oligonucleotide are administered before 3 doses of the HBV antigen vaccine. In exemplary embodiments, 3 doses of the oligonucleotide are administered before 4 doses of the HBV antigen vaccine.

In some embodiments, the oligonucleotide is administered 1, 2, 3, 4, 5, 6, 7, 8, 9, 10, 11, 12, 13, 14, 15, 16, 17, 18, 19, or 20 days before the HBV antigen vaccine is administered. In some embodiments, the oligonucleotide is administered 1, 2, 3, 4, 5, 6, 7, or 8 weeks before the HBV antigen vaccine is administered.

In some embodiments, a single one time dose of the oligonucleotide/HBV antigen vaccine of the disclosure is administered to a subject. In other embodiments, multiple doses are administered to the subject. In some embodiments, administration of the oligonucleotide/HBV antigen vaccine of the disclosure can comprise a dosing schedule in which they are administered more frequently initially, followed by less frequent doses. In other embodiments, administration of the oligonucleotide/HBV antigen vaccine of the disclosure can comprise a dosing schedule in which they are at different dosages at different intervals.

In some embodiments, the oligonucleotide in the vaccine is administered to a subject in a dose ranging from about 1 mg to about 1000 mg, 1 mg to 500 mg, 1 mg to 250 mg, 1 mg to 150 mg, 1 mg to 100 mg, 1 mg to 50 mg, 1 mg to 25 mg, 10 mg to 1000 mg, 10 mg to 500 mg, 10 mg to 250 mg, 10 mg to 150 mg, 10 mg to 100 mg, 10 mg to 50 mg, 10 mg to 50 mg, 10 mg to 25 mg, 50 mg to 1000 mg, 50 mg to 500 mg, 50 mg to 250 mg, 50 mg to 150 mg, 50 mg to 100 mg, 50 mg to 75 mg, 100 mg to 1000 mg, 100 mg to 500 mg, 100 mg to 400 mg, 100 mg to 300 mg, 100 mg to 250 mg, 100 mg to 200 mg, 100 mg to 150 mg, 200 mg to 500 mg, 200 mg to 250 mg, 300 mg to 1000 mg, or about 300 mg to 500 mg.

In some embodiments, the HBV antigen in the vaccine is administered to a subject in a dose ranging from about 1 μg to about 500 μg, per dose, about 3 μg to about 200 μg, about 3 μg to about 100 μg, about 3 μg to about 50 μg, about 3 μg to about 30 μg, about 3 μg to about 20 μg, about 3 μg to about 10 μg, about 5 μg to about 100 μg, about 5 μg to about 75 μg, about 5 μg to about 50 μg, about 5 μg to about 40 μg, about 5 μg to about 30 μg, about 5 μg to about 20 μg, about 5 μg to about 10 μg, about 10 μg to about 100 μg, about 10 μg to about 75 μg, about 10 μg to about 50 μg, about 10 μg to about 40 μg, about 10 μg to about 30 μg, about 10 μg to about 20 μg, about 20 μg to about 100 μg, about 50 μg to about 200 μg, or about 100 μg to about 500 μg.

In some embodiments, the oligonucleotide/HBV antigen vaccine of the disclosure, or pharmaceutical composition comprising the same are administered to a subject daily, every 2 days, every 3 days, every 4 days, every 5 days, every 6 days, every 7 days, every 8 days, every 9 days, every 10 days, every 11 days, every 12 days, every 13 days, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 2 months, every 3 months or every 4 months, or some combination thereof. It is noted that the intervals between doses may not be equal, and that the disclosure contemplates varying intervals between dosing.

In some embodiments, the oligonucleotide/HBV antigen vaccine of the disclosure, or pharmaceutical composition comprising the same are administered to a subject for at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, at least 2 years, at least 3 years, at least 4 years, or at least 5 years.

In the therapeutic vaccine context, in some embodiments, one or more pretreatments may precede the administration of the oligonucleotide/HBV antigen vaccine of the disclosure. Without being held to theory or mechanism, a pretreatment can work to decrease the levels of the HBV titer, e.g. as assayed by the reduction of levels of one or more HBV proteins or antigens. As shown in the examples, however, a pretreatment is not necessary, but in some embodiments, could lead to less variability in response. It is to be understood that treatment may occur in cycles, and each cycle may be preceded by a pretreatment.

The pretreatment may comprise administration of one or more oligonucleotides of the disclosure, e.g. an oligonucleotide comprising a sequence of any one of SEQ ID NOS: 10-666, or a modified oligonucleotide comprising 1, 2, 3, 4, 5, 6, 7, 8, 9, or 10 sequence modifications thereto, or an oligonucleotide comprises the sequence of any one of SEQ ID NOS: 10 to 666, or an oligonucleotide comprising at least 60%, 65%, 70%, 75%, 80%, 85%, 90%, 95% or 99% sequence identity thereto.

The pretreatment may comprise or ore more administrations of an HBV treatment, for example, an oligonucleotide, an antisense oligonucleotide, a siRNA directed to HBV, Interferon gamma, a PD-1 inhibitor, PD-L1 inhibitor, or other known HBV agent. In some embodiments, the pretreatment is before the first dose of vaccine. In some embodiments, the pretreatment is both before the first dose of vaccine and after the first dose or subsequent doses of vaccine if additional cycles of treatment are provided, as noted above.

Exemplary, non-limiting vaccine dosing schedules are provided for in Table 3. Each of these exemplary schedules may be preceded by a pretreatment as described herein. Each of these exemplary schedules can represent one cycle, and the cycle can be repeated a future time, optionally preceded by a pretreatment.

TABLE 3
Exemplary Vaccine Dosing Schedules

| 1^{st} Dose | 2^{nd} Dose | 3^{rd} Dose | 4^{th} Dose | 5^{th} Dose | 6^{th} Dose |
|---|---|---|---|---|---|
| Vaccine | Vaccine | Vaccine | Vaccine | Vaccine | Vaccine |
| Day 1 | Day 8 | Day 22 | | | |
| Day 1 | Day 8 | Day 29 | | | |
| Day 1 | Day 15 | Day 29 | | | |
| Day 1 | Day 22 | Day 43 | | | |
| Day 1 | Day 15 | Day 29 | Day 57 | | |
| Day 1 | Day 15 | Day 29 | Day 43 | Day 57 | |
| Day 1 | Day 8 | Day 15 | Day 22 | Day 29 | |
| Day 1 | Day 15 | Day 29 | Day 57 | Day 85 | |
| Day 1 | Week 2 | Week 4 | Week 8 | Week 16 | |
| Day 1 | Day 15 | Day 29 | Day 43 | Day 57 | Day 71 |

In the therapeutic vaccine context, in some embodiments, a therapeutically effective dose comprises a dose at which a maximum log 10 serum HBsAg (or other HBV protein) reduction of a least −0.15, at least −0.2, at least −0.3, at least −0.4, at least −0.5, at least −0.7, at least −1.0, at least −1.5, at least −2.0, at least −2.5, at least −3.0, at least −3.5, at least at least −4.0, or at least −4.5 is observed. In some embodiments, the reduction in maximum log 10 serum HBsAg (or other HBV protein) is observed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days after administration of the therapeutically effective dose.

In the therapeutic vaccine context, in some embodiments, a therapeutically effective dose of the oligonucleotide/HBV antigen vaccine of the disclosure, or pharmaceutical composition comprising the same, comprises a dose that decreases a level of HBsAg (or other HBV protein) in serum of the subject by at least about 10%, 20%, 30%, 40%, 50%, 60%, 70%, 80%, 90%, 95%, 99%, 99.7%, 99.9%, 99.97% %, 99.99% or 99.997%, or a range defined by any two of these values. In some embodiments, the reduction in serum HBsAg (or other HBV protein) is observed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days after administration of the therapeutically effective dose.

In the therapeutic vaccine context, in some embodiments, the oligonucleotide/HBV antigen vaccine of the disclosure, or pharmaceutical composition comprising the same are administered to the subject until a specific outcome is achieved, for example the level of HBsAg and/or HBeAg in the serum of the subject is decreased. In some embodiments, administration of the oligonucleotide/HBV antigen vaccine of the disclosure to the subject continues until the HBsAg and/or HBeAg in the serum of the subject is decreased by at least about 10%, 20%, 30%, 40%, 50%, 60%, 70%, 80%, 90%, 95%, 99%, 99.7%, 99.9%, 99.97% %, 99.99% or 99.997%, or a range defined by any two of these values, compared to before the administration.

In some embodiments, administration of the oligonucleotide/HBV antigen vaccine of the disclosure comprises a dosing holiday. Dosing holidays of certain duration and frequency are contemplated as within the scope of the disclosure. For example, the oligonucleotide/HBV antigen vaccine of the disclosure, or pharmaceutical composition comprising the same may be administered to the subject until levels of HBsAg fall below a certain threshold, or no HBV infection is detected in the subject, followed by a dosing holiday, and resumption of dosing if HBsAg in the serum of the subject is again detected.

e. Combination Therapies

In some embodiments the vaccines of the disclosure are designated as a first agent, and the methods comprise administering a first agent and one or more second agents. In some embodiments, the first agent and one or more second agents are co-administered. In some embodiments the first agent and one or more second agents are co-administered sequentially or simultaneously. In some embodiments, the first agent and one or more second agents are not co-administered.

In some embodiments, the oligonucleotide, the HBV antigen and the second agent are prepared together in a single formulation. In other embodiments, they are prepared separately.

The second agents of the disclosure may be administered by the same or by a different route of administration as the first agent vaccine.

For clarity by using the term “first” and “second” does not imply the order of administration. Thus in some embodiments, the first agent vaccine may be administered first, followed by one or more second agents of the disclosure. In other embodiments, the first agent vaccine and the second agent are administered in the reverse order.

In some embodiments, the second agent is designed to treat the same disease, disorder, or condition as the one or more vaccines provided herein. In other embodiments, the second agent is designed to treat a different disease, disorder, or condition as the one or more vaccines provided herein. In some embodiments, the second agent is designed to treat an undesired side effect of the one or more vaccines provided herein. In some embodiments, the one or more second agents provided herein are co-administered with the first agent vaccine to produce a combinational effect. In some embodiments, the one or more second agents provided herein are co-administered with the first agent vaccine to produce a synergistic effect.

In some embodiments, the disease comprises liver disease, and the second agent comprises lamivudine, entecavir, telbivudine, tenofovir, Adefovir, or Tenofovir alafenamide (TAF). In some embodiments, the second agent comprises Interferon alpha, Myrcludex B, Amantadine, Famciclovir, Prednisone, or more generally, corticosteroids, diuretics, beta-blockers, or a combination thereof. Examples of second agents can also include, but are not limited to, an anti-inflammatory agent, chemotherapeutic agent or anti-infection agent. In some embodiments, the condition comprises liver cancer, and the second agent comprises a chemotherapeutic agent, such as Gemcitabine (Gemzar), Oxaliplatin (Eloxatin), Cisplatin, Doxorubicin, 5-Fluorouracil, Capecitabine (Xeloda), or Mitoxantrone (Novantrone).

Due to overlapping transmission routes, many people have been exposed to both hepatitis B virus (HBV) and hepatitis C virus (HCV), and a smaller proportion are chronically infected with both viruses, especially in regions such as Asia where HBV is endemic. Estimates suggest that up to 10% of people with HCV may also have HBV, while perhaps 20% of people with HBV are co-infected with HCV. However, treatment of hepatitis B or hepatitis B in I-IBV-HCV co-infected individuals has not been well studied. Treatment is complicated by the fact that HCV and HBV appear to inhibit each other's replication (though not all studied have observed this interaction). Therefore, treatment that fully suppresses HBV could potentially allow HCV to re-emerge, or vice versa. Therefore, the vaccines described herein may advantageously be used for treating patients infected with both HBV and HCV when administered with a HCV-specific second agent. Exemplary treatment options for hepatitis C (HCV) include interferons, e.g., interferon alpha-2b, interferon alpha-2a, and interferon alphacon-1. Less frequent interferon dosing can be achieved using pegylated interferon (interferon attached to a polyethylene glycol moiety which improves its pharmacokinetic profile). Combination therapy with interferon alpha-2b (pegylated and unpegylated) and ribavirin has also been shown to be efficacious for some patient populations. Other agents currently being developed include HCV RNA replication inhibitors (e.g., ViroPharma's VP50406 series), HCV antisense agents, HCV therapeutic vaccines, HCV protease inhibitors, HCV helicase inhibitors and HCV antibody therapy (monoclonal or polyclonal). Due to overlapping transmission routes, many people have been exposed to also hepatitis D virus (HDV).

### III. Pharmaceutical Compositions

The disclosure provides pharmaceutical compositions comprising the vaccines of the disclosure and a pharmaceutically acceptable carrier, diluent or excipient.

In some embodiments, a pharmaceutical composition comprises an oligonucleotide of the disclosure, a HBV antigen, and a pharmaceutically acceptable carrier, diluent or excipient.

In other embodiments, one pharmaceutical composition comprises an oligonucleotide of the disclosure and a pharmaceutically acceptable carrier, diluent or excipient; and a second pharmaceutical composition comprises a HBV antigen of the disclosure and a pharmaceutically acceptable carrier, diluent or excipient, and the two pharmaceutical compositions are administered sequentially or simultaneously, or are mixed just prior to administration.

A variety of routes are contemplated for administration, as noted here, including oral, pulmonary, rectal, parenteral, transdermal, subcutaneous, intravenous, intramuscular, intraperitoneal, inhalational, buccal, sublingual, intrapleural, intrathecal, intranasal, via the CSF, and the like. A pharmaceutical composition of the disclosure is formulated to be compatible with its intended route of administration.

Dosage forms for the topical or transdermal administration of a of this invention include powders, sprays, ointments, pastes, creams, lotions, gels, solutions, patches and inhalants. In some embodiments, the modified oligonucleotide is mixed under sterile conditions with a pharmaceutically acceptable carrier/diluent/excipient, and with any preservatives, buffers, or propellants that are required.

Solutions or suspensions used for parenteral, intradermal, intraperitoneal or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral or subcutaneous preparation can be enclosed in ampoules, disposable syringes or multiple dose vials made of glass or plastic. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules, syringes and vials, and can be stored in a freeze-dried (lyophilized) condition requiring only the addition of the sterile liquid carrier, for example, saline or water-for-injection immediately prior to use.

The pharmaceutical composition described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active agents into preparations that can be used pharmaceutically. The appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible pharmaceutically acceptable carrier. They can be enclosed in gelatin capsules or compressed into tablets. For the purpose of oral therapeutic administration, the active age can be incorporated with excipients and used in the form of tablets, troches, or capsules. Oral compositions can also be prepared using a fluid carrier for use as a mouthwash, wherein the agents in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the agents are delivered in the form of an aerosol spray from pressured container or dispenser, which contains a suitable propellant, e.g., a gas such as carbon dioxide, or a nebulizer.

Pharmaceutical compositions can be prepared with pharmaceutically acceptable carriers that protect the oligonucleotides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art, and the materials can be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It may be desirable to formulate oral or parenteral compositions in dosage unit form for ease of administration and uniformity of dosage. Dosage unit form as used herein refers to physically discrete units suited as unitary dosages for the subject to be treated; each unit containing a predetermined quantity of the modified oligonucleotide calculated to produce the desired therapeutic effect in association with the required pharmaceutical carrier. The specification for the dosage unit forms of the disclosure are dictated by and directly dependent on the unique characteristics of the active agent and the particular therapeutic effect to be achieved.

The pharmaceutical compositions can be included in a container, pack, or dispenser together with instructions for administration.

Examples of pharmaceutically acceptable salts include, but are not limited to, mineral or organic acid salts of basic residues such as amines, alkali or organic salts of acidic residues, and the like. The pharmaceutically acceptable salts include the conventional sodium salt, calcium salt, magnesium salt, or other non-toxic salts or the quaternary ammonium salts of the parent compound formed, for example, from non-toxic inorganic or organic acids.

Techniques for formulation and administration of the disclosed compositions of the invention can be found in Remington: the Science and Practice of Pharmacy, 19th edition, Mack Publishing Co., Easton, PA (1995).

The oligonucleotides of the disclosure can be encapsulated within, or incorporated on the surface of particles. In some embodiments, the particle is a nanoparticle. Exemplary nanoparticles include liposomes, micelles, polymer-based nanoparticles, lipid-polymer based nanoparticles, and polymeric micelles.

In some embodiments, the nanoparticle comprises a liposome. Liposomes are spherical vesicles having at least one lipid bilayer, and in some embodiments, an aqueous core. In some embodiments, the lipid bilayer of the liposome may comprise phospholipids. An exemplary but non-limiting example of a phospholipid is phosphatidylcholine, but the lipid bilayer may comprise additional lipids, such as phosphatidylethanolamine. Liposomes may be multilamellar, i.e. consisting of several lamellar phase lipid bilayers, or unilamellar liposomes with a single lipid bilayer. Liposomes can be made in a particular size range that makes them viable targets for phagocytosis. Liposomes can range in size from 20 nm to 100 nm, 100 nm to 400 nm, 1 μM and larger, or 200 nm to 3 μM. Examples of lipidoids and lipid-based formulations are provided in U.S. Published application No. 20090023673. In other embodiments, the one or more lipids are one or more cationic lipids. One skilled in the art will recognize which liposomes are appropriate for encapsulation of the modified oligonucleotides described herein.

In some embodiments, the nanoparticle comprises a micelle. A micelle is an aggregate of surfactant molecules. An exemplary micelle comprises an aggregate of amphiphilic macromolecules, polymers or copolymers in aqueous solution, wherein the hydrophilic head portions contact the surrounding solvent, while the hydrophobic tail regions are sequestered in the center of the micelle.

In some embodiments, the nanoparticle comprises a polymer based nanoparticle. Polymer based nanoparticles comprise one or more polymers, such as a polyester, poly(orthoester), poly(ethylene imine), poly(caprolactone), polyanhydride, poly(acrylic acid), polyglycolide or poly(urethane). In still other embodiments, the one or more polymers comprise poly(lactic acid) (PLA) or poly(lactic-co-glycolic acid) (PLGA). In exemplary embodiments, the one or more polymers comprise polyalkylene glycol such as polyethylene glycol (PEG), or polyalkylene oxide, such as polyethylene oxide (PEO).

In some embodiments, the nanoparticles or some portion thereof are degradable. In other embodiments, the lipids and/or polymers of the nanoparticles are degradable.

In some embodiments, pharmaceutical compositions comprising an oligonucleotide and/or a HBV antigen are used for the preparation of a medicament for treating a patient suffering or susceptible to an HBV infection and/or a HBV-related condition.

### IV. Kits and Articles of Manufacture

The disclosure provides kits and articles of manufacture comprising the vaccines described herein, and pharmaceutical compositions comprising same. In some embodiments, the at least one oligonucleotide and the at least one HBV antigen are provided in the same vial. In some embodiments, the at least one oligonucleotide and the at least one HBV antigen are provided in two separate vials, either for mixing at bedside, or for separate administration. In some embodiments, the oligonucleotide comprises a sequence of any one of SEQ ID NOS: 10-666, or a modified oligonucleotide comprising 1, 2, 3, 4, 5, 6, 7, 8, 9, or 10 sequence modifications thereto. In some embodiments, the oligonucleotide comprises the sequence of any one of SEQ ID NOS: 10 to 666, or an oligonucleotide comprising at least 60%, 65%, 70%, 75%, 80%, 85%, 90%, 95% or 99% sequence identity thereto. In some embodiments, the oligonucleotide is modified.

The kit can further include instructions or a label for using the kit to treat, prevent, or ameliorate the HBV-related disease, disorder or condition.

Compositions comprising oligonucleotides and/or HBV antigens can be lyophilized before being packaged in the kit, or can be provided in solution or suspension with a pharmaceutically acceptable carrier, diluent, or excipient.

In some embodiments, the kit further comprises at least one additional agent for the treatment of an HBV-related disease, disorder or condition, as described herein.

### V. Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification at the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“5-methylcytosine” means a cytosine modified with a methyl group attached to the 5 position. A 5-methylcytosine is a modified nucleobase.

“About” means within ±10% of a value. For example, if it is stated, “the oligonucleotides affected at least about 70% inhibition of target”, it is implied that the target levels are inhibited within a range of 60% and 80%.

“100% complementary” means each nucleobase of a first nucleic acid has a complementary nucleobase in a second nucleic acid. In certain embodiments, a first nucleic acid is an oligonucleotide and a target nucleic acid is a second nucleic acid.

“Acute hepatitis B infection” results when a person exposed to the hepatitis B virus begins to develop the signs and symptoms of viral hepatitis. This period of time, called the incubation period, is an average of about 90 days, but could be as short as about 45 days or as long as about 6 months.

“Animal” refers to a human or non-human animal, including, but not limited to, mice, rats, rabbits, dogs, cats, pigs, primates, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanose ring modified by the bridging of two non-geminal carbon atoms. A bicyclic sugar is a modified sugar.

“Cap structure” or “terminal cap moiety” means chemical modifications, which have been incorporated at either terminus of an antisense compound.

“cEt” or “constrained ethyl” means a bicyclic sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an oligonucleotide that is in some way chemically different than another region of the same oligonucleotide. For example, a region having 2′-O-methoxyethyl nucleotides is chemically distinct from a region having nucleotides without 2′-O-methoxyethyl modifications.

“Chronic hepatitis B infection” occurs when a person initially suffers from an acute infection but is then unable to fight off the infection. Whether the disease becomes chronic or completely resolves depends mostly on the age of the infected person.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses administration in parallel or sequentially.

“Complementarity” means the capacity for pairing between nucleobases of a first nucleic acid and a second nucleic acid.

“Diluent” means an ingredient in a composition that lacks pharmacological activity, but is pharmaceutically necessary or desirable. For example, in drugs that are injected, the diluent may be a liquid, e.g. saline solution.

“Dosage unit” means a form in which a pharmaceutical agent is provided, e.g. pill, tablet, or other dosage unit known in the art.

“Dose” means a specified quantity of a pharmaceutical agent provided in a single administration, or in a specified time period. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In other embodiments, the pharmaceutical agent is administered by infusion over an extended period of time or continuously. Doses may be stated as the amount of pharmaceutical agent per hour, day, week or month.

“Duration” means the period of time during which an activity or event continues. In certain embodiments, the duration of treatment is the period of time during which doses of a pharmaceutical agent are administered.

A “gap” is an internal segment of an oligonucleotide that comprises one or more linked deoxynucleosides, and is positioned directly or indirectly between a 5′ wing (W1) and a 3′ wing (W2). A gap may be interchangeably referred to as a “gap” a “gap region” or a “gap segment”.

“HBV” means mammalian hepatitis B virus, including human hepatitis B virus. The term encompasses geographical genotypes of hepatitis B virus, particularly human hepatitis B virus, as well as variant strains of geographical genotypes of hepatitis B virus.

“HBV antigen” means any hepatitis B virus antigen or protein, including core proteins such as “hepatitis B core antigen” or “HBcAG” and “hepatitis B E antigen” or “HBeAg” and envelope proteins such as “HBV surface antigen”, or “HBsAg” or “HBsAG”.

“Hepatitis B E antigen” or “HBeAg” or “HBeAg” is a secreted, non-particulate form of HBV core protein. HBV antigens HBeAg and HBcAg share primary amino acid sequences, so show cross-reactivity at the T cell level.

“HBV surface antigen”, or “HBsAg”, or “HBsAg” is the envelope protein of infectious HBV viral particles but is also secreted as a non-infectious particle with serum levels higher than HBV viral particles.

“Individual” means a human or non-human animal selected for treatment or therapy.

“Induce”, “inhibit”, “potentiate”, “elevate”, “increase”, “decrease” or the like, generally denote quantitative differences between two states. Such terms may refer to a statistically significant difference between the two states. Such terms are applied to, for example, levels of expression, and levels of activity. The term “inhibit” or “reduce” or grammatical variations thereof, as used herein, refer to a decrease or diminishment in the specified level or activity of at least about 5%, about 10%, about 15%, about 25%, about 35%, about 40%, about 50%, about 60%, about 75%, about 80%, about 90%, about 95% or more. In some embodiments, the inhibition or reduction results in little or essentially no detectible activity (at most, an insignificant amount, e.g., less than about 10% or even 5%).

“Intraperitoneal administration” means administration through infusion or injection into the peritoneum.

“Intravenous administration” means administration into a vein.

“Inhibiting the expression or activity” refers to a reduction, blockade of the expression or activity and does not necessarily indicate a total elimination of expression or activity.

“Linked deoxynucleoside” means a deoxyribonucleic acid base (A, G, C, T, U) linked by a phosphate ester to form a nucleotide.

“Linked nucleosides” means adjacent nucleosides linked together by an internucleoside linkage. An example of linked nucleoside is a linked nucleotide in which the linkage involves a phosphate group atom, e.g. a phosphodiester bond.

“Locked nucleic acid” or “LNA” or “LNA nucleosides” means nucleic acid monomers having a bridge connecting two carbon atoms between the 4′ and 2′ position of the nucleoside sugar unit, thereby forming a bicyclic sugar. Examples of such bicyclic sugar include, but are not limited to A) α-L-Methyleneoxy (4′-CH₂—O-2′) LNA; (B) β-D-Methyleneoxy (4′-CH₂—O-2′)-LNA; (C) Ethyleneoxy (4′-(CH₂)₂—O-2′) LNA; (D) Aminooxy (4′-CH₂—O—NI-2′) LNA; and (E) Oxyamino (4′-CH₂—NI—O-2′) LNA; as depicted below.

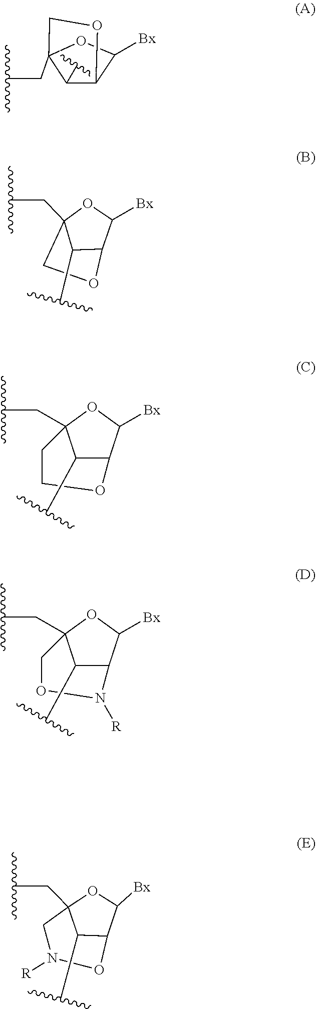

As used herein, LNA oligonucleotides include, but are not limited to, oligonucleotides having at least one bridge between the 4′ and the 2′ position of the sugar wherein each of the bridges independently comprises 1 or from 2 to 4 linked groups independently selected from —[C(R¹)(R²)]n-, —C(R¹)═C(R²)—, —C(R¹)═N—, —C(═NR¹)—, —C(═O)—, —C(═S)—, —O—, —Si(R¹)₂—, —S(═O)ₓ— and —N(R¹)—; wherein: x is 0, 1, or 2; n is 1, 2, 3, or 4; each R¹ and R² is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ¹, NJ¹J², SJ¹, N₃, COOJ¹, acyl (C(═O)—H), substituted acyl, CN, sulfonyl (S(═O)₂-J¹), or sulfoxyl (S(═O)-J¹); and each J¹ and J² is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl(C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R¹)(R²)]ₙ—, —[C(R¹)(R²)]ₙ—O—, —C(R¹)(R²)—N(R¹)—O— or —C(R¹)(R²)—O—N(R¹)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R¹)-2′ and 4′-CH₂—N(R¹)—O-2′-bridges, wherein each R¹ and R² is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′-CH₂—O-2′) bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used. A-L-methyleneoxy (4′-CH₂—O-2′), an isomer of methyleneoxy (4′-CH₂—O-2′) LNA is also encompassed within the definition of LNA, as used herein.

“Modified internucleoside linkage” refers to a substitution or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase.

“Modified sugar” means substitution and/or any change from a natural sugar moiety.

“Monomer” refers to a single unit of an oligomer. Monomers include, but are not limited to, nucleosides and nucleotides, whether naturally occurring or modified.

“Motif” means the pattern of unmodified and modified nucleosides in an antisense oligonucleotide. “Natural sugar moiety” means a sugar moiety found in DNA (2′-H) or RNA (2′-OH). “Naturally occurring internucleoside linkage” means a 3′ to 5′ phosphodiester linkage.

“Nucleobase” means a nitrogenous heterocyclic base moiety capable of pairing with a base of another nucleic acid.

“Nucleobase sequence” means the order of contiguous nucleobases independent of any sugar, linkage, and/or nucleobase modification.

“Nucleoside” means a nucleobase linked to a sugar. A nucleoside includes deoxynucleosides, e.g. deoxyribonucleosides.

“Nucleotide” means a nucleoside having a phosphate group covalently linked to the sugar portion of the nucleoside.

“Oligonucleotide” means a polymer of linked nucleosides, through internucleoside linkages, each of he linked nucleosides can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Pharmaceutically acceptable carrier” means a medium or diluent that does not interfere with the structure of the oligonucleotide. Certain such carriers enable pharmaceutical compositions to be formulated as, for example, tablets, pills, dragees, capsules, liquids, gels, syrups, slurries, suspension and lozenges for the oral ingestion by a subject.

“Pharmaceutically acceptable derivative” encompasses pharmaceutically acceptable salts, conjugates, prodrugs or isomers of the oligonucleotides described herein.

“Pharmaceutically acceptable salts” means physiologically and pharmaceutically acceptable salts of antisense oligonucleotides, i.e., salts that retain the desired biological activity of the parent oligonucleotide and do not impart undesired toxicological effects thereto.

“Pharmaceutical agent” means a substance that provides a therapeutic benefit when administered to an individual.

“Pharmaceutical composition” means a mixture of substances suitable for administering to a subject. For example, a pharmaceutical composition may comprise an antisense oligonucleotide and a sterile aqueous solution. In certain embodiments, a pharmaceutical composition shows activity in free uptake assay in certain cell lines.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Prevention” or “preventing” refers to delaying or forestalling the onset or development of a condition or disease for a period of time from hours to days, preferably weeks to months.

“Prophylactically effective amount” refers to an amount of a pharmaceutical agent that provides a prophylactic or preventative benefit to an animal.

“Ribonucleotide” means a nucleotide having a hydroxy group at the 2′ position of the sugar portion of the nucleotide. Ribonucleotides may be modified with any of a variety of substituents.

“Salts” mean a physiologically and pharmaceutically acceptable salts of antisense oligonucleotides, i.e., salts that retain the desired biological activity of the parent oligonucleotide and do not impart undesired toxicological effects thereto.

“Segments” may be interchangeably referred to as “regions” or “portions”.

A “separator” in the oligonucleotides of the disclosure is positioned directly in between, and separates two gap regions and is positioned in between two gap regions in an oligonucleotide. The separator may have one or more nucleosides, wherein the nucleosides are chemically distinct from the nucleosides comprising the gap. A separator segment comprises nucleosides modified to impart properties such as enhanced inhibitory activity, increased binding affinity for a target nucleic acid, lowered in vivo toxicity or resistance to degradation by in vivo nucleases. An oligonucleotide may comprise one or more separator segments. Exemplary oligonucleotides of the disclosure may comprise one, two, three, four, five or six separator segments. In some embodiments, the oligonucleotides comprise one separator segment. In some embodiments, the oligonucleotides comprise two separator segments.

“Seroconversion” is defined as serum HBeAg absence plus serum HBeAb presence, if monitoring HBeAg as the determinant for seroconversion, or defined as serum HBsAg absence, if monitoring HBsAg as the determinant for seroconversion, as determined by currently available detection limits of commercial ELISA systems.

“Target nucleic acid,” “target RNA,” “target RNA transcript” and “nucleic acid target” all mean a nucleic acid capable of being targeted by antisense oligonucleotides.

“Target region” means a portion of a target nucleic acid to which one or more antisense oligonucleotides is targeted.

“Target segment” means the sequence of nucleotides of a target nucleic acid to which an antisense oligonucleotide is targeted.

“Therapeutically effective amount” means an amount of a pharmaceutical agent that provides a therapeutic benefit to an individual.

“Treatment” refers to administering a composition to effect an alteration or improvement of the disease or condition.

“Unmodified” nucleobases mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. (3-D-ribonucleosides) or a DNA nucleotide (i.e. (3-D-deoxyribonucleoside).

“Validated target segment” is defined as at least an 8-nucleobase portion (i.e. 8 consecutive nucleobases) of a target region to which an active oligomeric oligonucleotide is targeted.

A “wing” is a terminal segment of an oligonucleotide, modified to impart to an oligonucleotide, properties such as enhanced inhibitory activity, enhanced biological activity, increased binding affinity for a target nucleic acid, lowered in vivo toxicity or resistance to degradation by in vivo nucleases. A wing may be referred to as a “wing” a “wing region” or a “wing segment”. As used herein, a wing comprises at least two linked nucleosides; a subset of which may comprise one or more deoxynucleosides, but the entire wing cannot be made up of only deoxynucleosides. In some embodiments, the wing comprises 2 to 8 linked nucleosides. In some embodiments, the wing comprises 2 to 6 linked nucleosides.

The oligonucleotides of the disclosure comprise a 5′ wing segment (W1) located at the 5′ terminus of the oligonucleotide and the residue at the 3′ end of the W1 is not a deoxynucleoside. The oligonucleotides of the disclosure also comprise a 3′ wing segment (W2) located at the 3′ terminus of the oligonucleotide and the 5′ terminal residue of the W2 is not a deoxynucleoside.

A 5′ wing (W1) begins at the 5′ terminus of the oligonucleotide, extends in the 5′ to 3′ direction, and that is directly linked to a deoxynucleoside of a first gap, thus indicating the 3′ end of the W1 and the 5′ end of a first gap (G1).

A 3′ wing (W2) begins at the 3′ terminus of the oligonucleotide, extends in the 3′ to 5′ direction, and that is directly linked to a deoxynucleoside of a gap, thus indicating the 3′ end of the last gap of and the 5′ end of the W2.

### ENUMERATED EMBODIMENTS

The following non-limiting enumerated embodiments are provided to illustrate embodiments of the invention.

Embodiment I-1. A vaccine composition comprising at least one oligonucleotide and at least one hepatitis B virus (HBV) antigen.

Embodiment I-2. The vaccine composition of embodiment I-1, wherein the oligonucleotide is complementary to one or more portions of the nucleic acid sequence of a HBV genome.

Embodiment I-3. The vaccine composition of embodiment I-1, wherein the oligonucleotide is not complementary to a portion of the nucleic acid sequence of a HBV genome.

Embodiment I-4. The vaccine composition of any one of embodiments I-2 to I-3, wherein the HBV genome is the genome of HBV GT-A, GT-B, GT-C, GT-D, GT-E, GT-F, GT-G, GT-H, GT-I, or GT-J.

Embodiment I-5. The vaccine composition of any one of embodiments I-1 to I-4, wherein the oligonucleotide comprises 10-60 nucleobases.

Embodiment I-6. The vaccine composition of any one of embodiments I-1 to I-5, wherein the oligonucleotide is a modified oligonucleotide.

Embodiment I-7. The vaccine composition of any one of embodiments I-1 to I-6, wherein the modified oligonucleotide comprises one or more modified sugars, modified bases, modified internucleoside linkages, or combinations thereof.

Embodiment I-8. The vaccine composition of any one of embodiments I-1 to I-6, wherein at least one internucleoside linkage is a phosphodiester linkage.

Embodiment I-9. The vaccine composition of any one of embodiments I-1 to I-8, wherein at least one internucleoside linkage is a modified internucleoside linkage.

Embodiment I-10. The vaccine composition of embodiment I-9, wherein the modified internucleoside linkage is a phosphorothioate internucleoside linkage.

Embodiment I-11. The vaccine composition of any one of embodiments I-1 to I-10, wherein the oligonucleotide comprises linked deoxynucleosides.

Embodiment I-12. The vaccine composition of any one of embodiments I-1 to I-11, wherein the oligonucleotide comprises at least one nucleoside comprising a modification to a C2′ position.

Embodiment I-13. The vaccine composition of embodiment I-12, wherein the oligonucleotide comprises at least one nucleoside comprising a 2′-O-methyl sugar modification, 2′-O-methoxyethyl sugar modification, 2′-fluoro sugar modification, and/or a 2′-fluoro-arabinonucleic acid (2′-fluoro-ANA) sugar modification.

Embodiment I-14. The vaccine composition of any one of embodiments I-1 to I-13, wherein the oligonucleotide comprises at least one bicyclic sugar modified nucleoside.

Embodiment I-15. The vaccine composition of any one of embodiments I-1 to I-14, wherein the oligonucleotide comprises at least one nucleoside comprising a phosphorodiamidate morpholino modification.

Embodiment I-16. The vaccine composition of any one of embodiments I-1 to I-15 wherein the oligonucleotide comprises at least one locked nucleic acid (LNA), peptide nucleic acid, and/or glycol nucleic acid.

Embodiment I-17. The vaccine composition of any one of embodiments I-1 to I-16, wherein the oligonucleotide is selected from SEQ ID NO: 10-666, or an oligonucleotide having at least 1 sequence modification thereto.

Embodiment I-18. The vaccine composition of any one of embodiments I-1 to I-17, wherein the oligonucleotide is selected from SEQ ID NO: 10-666, or an oligonucleotide having at least 70% sequence identity thereto.

Embodiment I-19. The vaccine composition of any one of embodiments I-1 to I-18, comprising at least two different oligonucleotides.

Embodiment I-20. The vaccine composition of any one of embodiments I-1 to I-19, wherein the HBV antigen is a human HBV antigen.

Embodiment I-21. The vaccine composition of any one of embodiments I-1 to I-20, wherein the HBV antigen is a HBV surface antigen (HBsAg), HBV e antigen (HBeAg), HBV core antigen (HBcAg), or any combination thereof.

Embodiment I-22. The vaccine composition of embodiment I-21, wherein the HBsAg is a Small-HBsAg, Medium-HBsAg, Large-HBsAg, or any combination thereof.

Embodiment I-23. The vaccine composition of any one of embodiments I-1 to I-22, wherein the HBV antigen is from a HBV GT-A, GT-B, GT-C, GT-D, GT-E, GT-F, GT-G, GT-H, GT-I, or GT-J genotype.

Embodiment I-24. The vaccine composition of any one of embodiments I-1 to I-23, wherein the HBV antigen is isolated from patients, recombinantly produced, or synthesized.

Embodiment I-25. The vaccine composition of any one of embodiments I-1 to I-24, comprising at least two different HBV antigens.

Embodiment I-26. The vaccine composition of any one of embodiments I-1 to I-25, wherein the vaccine composition comprises an adjuvant.

Embodiment I-27. The vaccine composition of embodiment I-26, wherein the adjuvant is an aluminum hydroxide.

Embodiment I-28. The vaccine composition of embodiment I-26, wherein the adjuvant is toll-like receptor 9 (TLR9) agonist.

Embodiment I-29. The vaccine composition of embodiment I-26, wherein the adjuvant is an oligonucleotide.

Embodiment I-30. The vaccine composition of embodiment I-27, wherein the oligonucleotide adjuvant comprises CpG1018.

Embodiment I-31. The vaccine composition of any one of embodiments I-1 to I-30, wherein the vaccine composition is a therapeutic vaccine composition.

Embodiment I-32. The vaccine composition of any one of embodiments I-1 to I-30, wherein the vaccine composition is a prophylactic vaccine composition.

Embodiment I-33. A pharmaceutical composition comprising the vaccine composition of any one of embodiments I-1 to I-32, and a pharmaceutically acceptable carrier, diluent, or excipient.

Embodiment I-34. A kit comprising the vaccine composition of any one of embodiments I-1 to I-32.

Embodiment I-35. A method of eliciting an immune response to a HBV antigen in a subject suffering from an HBV infection, or a HBV-related disease, disorder, or condition, comprising administering to the subject a vaccine composition of any one of embodiments I-1 to I-32, or the pharmaceutical composition of embodiment I-33.

Embodiment I-36. A method of treating or preventing an HBV infection, or a HBV-related disease, disorder, or condition in a subject with a vaccine treatment, comprising administering to the subject a vaccine composition of any one of embodiments I-1 to I-32, or the pharmaceutical composition of embodiment I-33.

Embodiment I-37. A method of eliciting an immune response to a HBV antigen in a subject suffering from an HBV infection, or a HBV-related disease, disorder, or condition with a vaccine treatment, comprising administering to the subject an oligonucleotide and a hepatitis B virus (HBV) antigen

Embodiment I-38. A method of treating or preventing an HBV infection, or a HBV-related disease, disorder, or condition in a subject with a vaccine treatment, comprising administering to the subject an oligonucleotide and a hepatitis B virus (HBV) antigen.

Embodiment I-39. The method of any one of embodiments I-37 to I-38, wherein the oligonucleotide and the HBV antigen are administered simultaneously.

Embodiment I-40. The method of embodiment I-39, wherein the oligonucleotide and the HBV antigen are provided in a premixture.

Embodiment I-41. The method of embodiment I-39, wherein the oligonucleotide and the HBV antigen are mixed just prior to administration.

Embodiment I-42. The method of embodiment I-39, wherein the oligonucleotide and the HBV antigen are simultaneously administered to different injection sites.

Embodiment I-43. The method of embodiment I-42, wherein the injection sites are adjacent.

Embodiment I-44. The method of embodiment I-39, wherein the oligonucleotide and the HBV antigen are simultaneously administered to the same injection site.

Embodiment I-45. The method of any one of embodiments I-37 to I-38, wherein the oligonucleotide and the HBV antigen are administered sequentially.

Embodiment I-46. The method of embodiment I-45, wherein the oligonucleotide and the HBV antigen are administered sequentially to the same injection site.

Embodiment I-47. The method of embodiment I-45, wherein the oligonucleotide and the HBV antigen are administered sequentially to different injection sites.

Embodiment I-48. The method of embodiment I-47, wherein the injection sites are adjacent.

Embodiment I-49. The method of embodiment I-45, wherein the oligonucleotide is administered before the HBV antigen.

Embodiment I-50. The method of embodiment I-49, wherein multiple doses of the oligonucleotide are administered before the HBV antigen is administered.

Embodiment I-51. The method of embodiment I-50, wherein 2, 3, 4, 5, or more doses of the oligonucleotide are administered before the HBV antigen is administered.

Embodiment I-52. The method of embodiment I-49, wherein a single dose of the oligonucleotide is administered before the HBV antigen is administered.

Embodiment I-53. The method of any one of embodiments I-49 to I-52, wherein multiple doses of the HBV antigen is administered after the oligonucleotide is administered.

Embodiment I-54. The method of any one of embodiments I-37 to I-53, wherein at least two doses of the oligonucleotide and/or HBV antigen are administered.

Embodiment I-55. The method of any one of embodiments I-37 to I-54, wherein the oligonucleotide comprises the oligonucleotide of one of embodiments I-2 to I-18.

Embodiment I-56. The method of any one of embodiments I-37 to I-55, wherein the HBV antigen is a HBV surface antigen (HBsAg), HBV e antigen (HBeAg), or HBV core antigen (HBcAg).

Embodiment I-57. The method of embodiment I-56, wherein the HBsAg is a Small-HBsAg, Middle-HBsAg, Large-HBsAg, or any combination thereof.

Embodiment I-58. The method of any one of embodiments I-37 to I-57, wherein the HBV antigen is from a HBV GT-A, GT-B, GT-C, GT-D, GT-E, GT-F, GT-G, GT-H, GT-I, or GT-J subtype.

Embodiment I-59. The method of any one of embodiments I-35 to I-58, wherein the method comprises one or pretreatments.

Embodiment I-60. The method of embodiment I-59, wherein the pretreatment comprises administering an oligonucleotide that is the same oligonucleotide as of the vaccine composition.

Embodiment I-61. The method of embodiment I-59, wherein the pretreatment comprises administering an oligonucleotide that is different from the oligonucleotide of the vaccine composition.

Embodiment I-62. The method of embodiment I-59, wherein the pretreatment comprises administering an HBV treatment selected from the group consisting of a siRNA directed to HBV, Interferon gamma, PD-1 inhibitor, and a PD-L1 inhibitor.

Embodiment I-63. The method of any one of embodiments I-35 to I-62, wherein the method results in a reduction of levels of HBsAg, HBcAg, and/or HBeAg in the serum.

Embodiment I-64. The method of any one of embodiments I-35 to I-63, wherein the method results in a reduction of levels of HBV DNA in the liver.

Embodiment I-65. The method of any one of embodiments I-35 to I-64, wherein the method results in a reduction of levels of HBV mRNA in the liver.

Embodiment I-66. The method of any one of embodiments I-35 to I-65, wherein the method results in an increase in levels of HBsAb in the serum.

Embodiment I-67. The method of any one of embodiments I-35 to I-66, wherein the method results in an increase a T cell response.

Embodiment I-68. The method of any one of embodiments I-63 to I-67, wherein the method results in dose-dependent effect.

Embodiment I-69. The method of any one of embodiments I-35 to I-68, wherein the subject is a primate.

Embodiment I-70. The method of embodiment I-69, wherein the subject is a human subject.

Embodiment I-71. The method of any one of embodiments I-35 to I-70, wherein the route of administration is subcutaneous, intramuscular, intravenous, or intradermal.

Embodiment I-72. The method of any one of embodiments I-35 to I-71, wherein the subject is suffering from an acute HBV infection.

Embodiment I-73. The method of any one of embodiments I-35 to I-71, wherein the subject is suffering from a chronic HBV infection.

Embodiment I-74. The method of any one of embodiments I-35 to I-73, wherein the subject is administered about 20 mg to about 500 mg per dose of the oligonucleotide.

Embodiment I-75. The method of any one of embodiments I-35 to I-74, wherein the subject is administered about 1 μg to about 500 μg per dose of the HBsAg.

Embodiment I-76. A kit comprising an oligonucleotide and a hepatitis B virus (HBV) antigen, of any of the above embodiments.

The following examples are included for illustrative purposes and are not intend to limit the scope of the invention.

### EXAMPLES

### Example 1: Immune Enhancing Activity of an Oligonucleotide in HBV Carrier Mice

It is noted that the oligonucleotides of the disclosure and these examples, and their respective AUS numbers, are provided in Tables 1.1 and 1.2, as presented for in FIG. 1 and FIG. 2 of the accompanying drawings. Oligonucleotides of the disclosure were synthesized using standard solid phase synthesis protocols, as described in International Publication No., WO2023131098A2.

The immune enhancing activity of an oligonucleotide of the disclosure, AUS1441, on the serum levels of Hepatitis B Surface antigen protein (HBsAg) and antibodies to the HBsAg (HBsAb) was evaluated in an HBV mouse model. The HBV mouse model was constructed by hydrodynamic-injection (HDI) of an HBV plasmid containing preS2/S of HBV sequence in C57BL/6 mice; the HBV carrier mice prepared in this way are referred to as HDI-HBV mice herein. The HDI-HBV mice were randomly divided into four groups of four animals each. In the first dosing period (a pretreatment period), all animals were dosed with AUS1463 at 60 mg/kg by subcutaneous injection (SC), in order to lower HBsAg levels. In the second dosing period (vaccine dosing period), which started at 10 days after dosing in the first period, the animals were subcutaneously dosed with 100 μl of a dosing solution comprised of one of the following: normal saline (Group A), 3 ug of HBsAg (Group B), 500 ug of AUS1441 and 3 ug of HBsAg (Group C), or 500 ug of AUS1441 (Group D). The start of the second dosing period (vaccine dosing period) was counted as Day 0. Each group was dosed on Day 0, Day 14, and Day 42 with the corresponding dosing solutions.

The AUS1463 dosing solution was prepared by dissolving AUS1463 powder in saline to a final concentration of 6 mg/ml. The AUS1441 working solution was prepared in saline at a concentration of 10 mg/ml. The HBsAg used in this example is a commercially available natural Hepatitis B surface antigen protein (purchased from Beijing Biolab Technology Co., Ltd), which has been isolated and purified from the plasma of chronic Hepatitis B patients. The HBsAg working solution was prepared in saline at a concentration of 0.06 mg/ml. The HBsAg working solution was mixed at a 1:1 volume ratio with the AUS1441 working solution (in Group C) approximately 30 min before administering to the animals. The dosing solutions for Group B and Group D were prepared by a two-fold dilution with saline of the working solutions of HBsAg and AUS1441, respectively.

Approximately 100 μl of blood sample was taken from a thigh vein of each animal each week. Blood samples were incubated at 37° C. for approximately 30 minutes, and then centrifuged in a precooled (0-4° C.) centrifuge to obtain the serum samples. The serum samples were stored at −20° C. until analysis. The serum HBsAg and HBsAb levels were measured by chemiluminescence immunoassay assays.

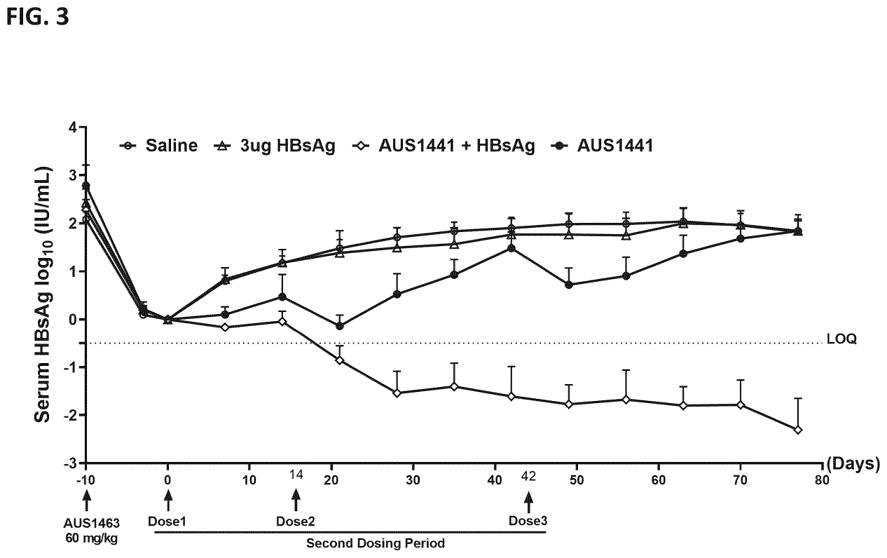

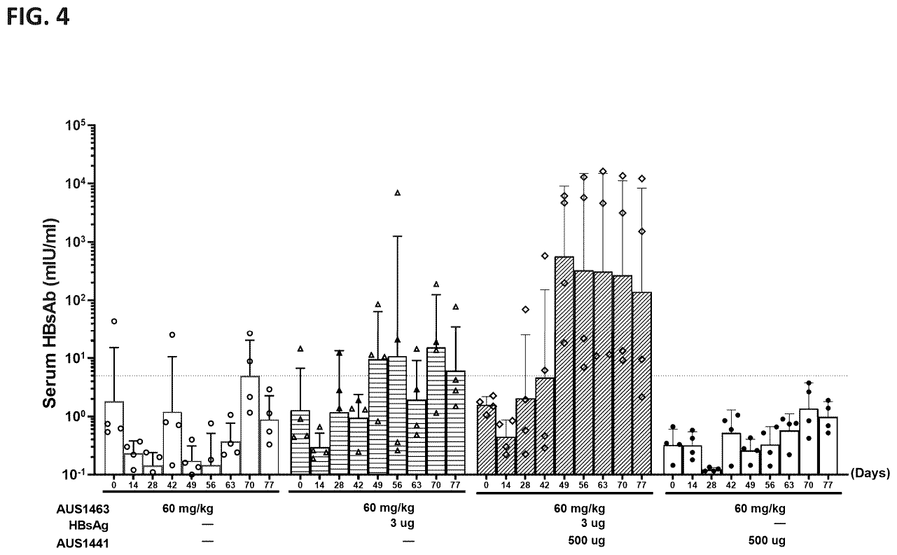

The serum HBsAg levels at different time points, normalized relative to the Day 0 level for each group, are shown in FIG. 3, the data points are the means and error bars are the standard deviation. The serum HBsAb levels in each group on Days 0, 14, 28, 42, 49, 56, 63, 70 and 77 are shown in FIG. 4, the boxes represent the geometric means, the error bars are the geometric standard deviation, and the dots are individual HBsAb levels.

Serum HBsAg and HBsAb levels on Day 77 are listed in Table 1.3. The serum HBsAg levels were decreased by >2 logs with a dose of 60 mg/kg AUS1463 in the HDI-HBV mice. In Group C (500 ug AUS1441+3 ug HBsAg), sustained serum HBsAg reduction below the limit of quantification (LOQ, which is 1 IU/ml for HBsAg) was achieved in all animals (4/4), and serum HBsAb levels were boosted in all animals in this group, which peaked on Day 49 with a geometric mean of 567 mIU/ml. When HBsAg (3 ug) was dosed alone (Group B), none of the animals in the group had HBsAg levels below the limit of quantification, and the HBsAb levels were generally low and variable with only one out the four animals had HBsAb level above the limit of quantification (LOQ, which is 5 mIU/ml for HBsAb) on Day 77. Similarly, when AUS1441 (500 ug) was dosed alone (Group D), none of the animals in the group had HBsAg levels below the limit of quantification, and the HBsAb levels were below the limit of quantification in all animals in the group (HBsAb LOQ=5 mIU/ml).

TABLE 1.3
Serum HBsAg and HBsAb levels
in HDI-HBV mice on Day 77

| | | HBsAg Level | HBsAb Level |
|---|---|---|---|
| Group | Dose | (IU/ml) | (mIU/ml) |
| A | Saline | >LOQ in 4/4 | <LOQ in all animals |
| | | animals, mean = | |
| | | 1.82 log10 | |
| B | 3 ug | >LOQ in 4/4 | >LOQ in 1/4 animal, |
| | HBsAg | animals, mean = | with an individual |
| | | 1.84 log10 | level of 77 mIU/ml |
| C | 500 ug | <LOQ* in 4/4 | >LOQ* in 3/4 animals, |
| | AUS1441 + | animals | geometric mean = |
| | 3 ug | | 139 mIU/ml, and max |
| | HBsAg | | individual level of |
| | | | 12190 mIU/ml |
| D | 500 ug | >LOQ in 4/4 | <LOQ in all animals |
| | AUS1441 | animals, mean = | |
| | | 1.84 log10 | |

*HBsAg LOQ = 1 IU/mL; HBsAb LOQ = 5 mIU/mL

### Example 2: Immune Enhancing Activity of Oligonucleotides in HBV Carrier Mice

The immune enhancing activity of the oligonucleotide presented in Example 1 are generalizable to other oligonucleotides of the disclosure, as shown in this example. The immune enhancing activity of oligonucleotides, including AUS1499, AUS1476, AUS1497, AUS1498, AUS1500, AUS1496, were evaluated in the HDI-HBV mice. The animals were randomly divided into eight groups of four animals each. In the first dosing period (pretreatment dosing period), all animals were administered with three weekly doses of AUS1233 at 60 mg/kg by subcutaneous injection (SC) to lower HBsAg levels. In the second dosing period (vaccine dosing period), which started at 21 days after the first dose in the first period, each group of the HDI-HBV mice were subcutaneously dosed with 100 μl of a dosing solution comprised of one of the following: 3 ug HBsAg (Group 1); a mixture containing 500 ug AUS1499 and 3 ug HBsAg (Group 2); a mixture containing 500 ug AUS1476 and 3 ug HBsAg (Group 3); a mixture containing 150 ug AUS1476 and 3 ug HBsAg (Group 4); a mixture containing 500 ug AUS1497 and 3 ug HBsAg (Group 5); a mixture containing 500 ug AUS1498 and 3 ug HBsAg (Group 6); a mixture containing 500 ug AUS1500 and 3 ug HBsAg (Group 7); or a mixture containing 500 ug AUS1496 and 3 ug HBsAg (Group 8). The start of the second dosing period was counted as Day 0. Each group was dosed on Day 0 and Day 14 with the corresponding dosing solutions.

The AUS1233 dosing solutions were prepared by dissolving AUS1233 powder in saline to make a concentration of 6 mg/ml. The working solutions of AUS1499, AUS1476, AUS1497, AUS1498, AUS1500 and AUS1496 were each prepared in saline at 10 mg/ml. In addition, a lower concentration working solution of AUS1476 was prepared in saline at 3 mg/ml. The HBsAg used in this example is a commercially available natural Hepatitis B surface antigen protein (purchased from Beijing Biolab Technology Co., Ltd), which has been isolated and purified from the plasma of chronic Hepatitis B patients. The HBsAg working solution was prepared in saline at a concentration of 0.06 mg/ml. The dosing solution for Group 1 was prepared by a two-fold dilution of HBsAg working solution with saline. The dosing solutions for Groups 2, 3, 5, 6, 7, and 8 were prepared by mixing the HBsAg working solution with the 10 mg/ml working solutions of AUS1499, AUS1476, AUS1497, AUS1498, AUS1500 and AUS1496, respectively, at a 1:1 volume ratio approximately 30 min before administering to the animals. The dosing solution for Group 4 was prepared by mixing the HBsAg working solution with the 3 mg/ml working solution of AUS1476 at a 1:1 volume ratio approximately 30 min before administering to the animals.

Approximately 100 μl of blood sample was taken from a thigh vein each week. Blood samples were incubated at 37° C. for approximately 30 minutes, and then centrifuged in a precooled (0-4° C.) centrifuge to obtain the serum samples. The serum samples were stored at −20° C. until analysis. The HBsAg and HBsAb levels was measured in the serum samples by Chemiluminescence immunoassay assays.

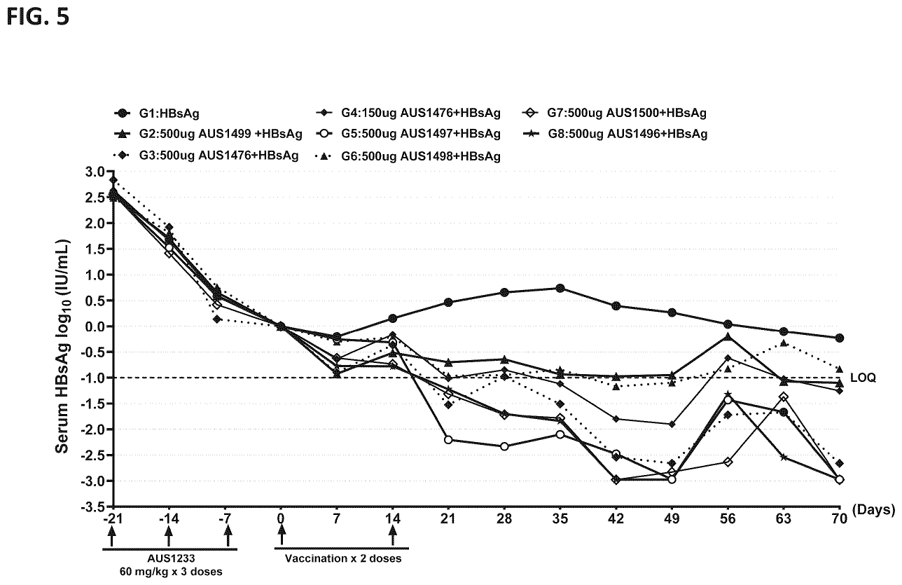

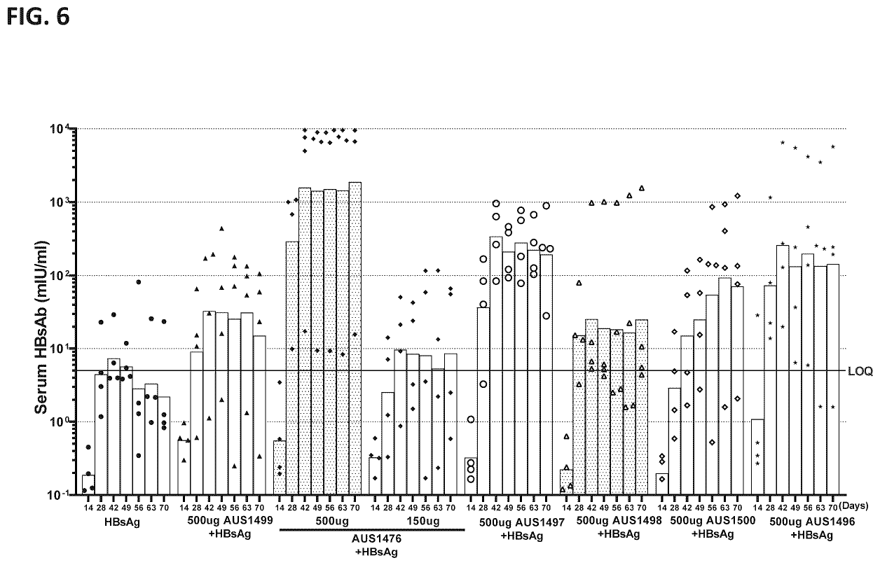

The serum HBsAg levels at different time points are normalized relative to the Day 0 level for each group, and are shown in FIG. 5, the data points represent the means. The serum HBsAb levels in each group on Days 14, 28, 42, 49, 56, 63, and 70 are shown in FIG. 6, the boxes represent the geometric means and the dots are individual HBsAb levels. Serum HBsAg and HBsAb levels on Day 70 are listed in Table 2.1. The serum HBsAg levels were decreased by >2 logs with three weekly subcutaneous doses of AUS1233 at 60 mg/kg to the HDI-HBV mice. When HBsAg (3 ug) alone was used (Group 1), the serum HBsAg level was only marginally decreased and the serum HBsAb was above the limit of quantification (LOQ, which is 5 mIU/ml for HBsAb) in just one animal out of the total of four. In Group 3 (500 ug AUS1476+3 ug HBsAg), Group 5 (500 ug AUS1497+3 ug HBsAg), Group 7 (500 ug AUS1500+3 μg HBsAg), and Group 8 (500 ug AUS1496+3 ug HBsAg), sustained serum HBsAg reduction below the limit of quantification (LOQ=1 IU/ml for HBsAg) was achieved in all animals (4/4), and serum HBsAb levels were boosted in all animals in each of these groups.

Dose dependent immune enhancing effect of oligonucleotides was observed, e.g. as exhibited by the results of Groups 3 and 4.

TABLE 2.1
Serum HBsAg and HBsAb levels in HDI-HBV mice on Day 70

| Group | Dose | HBsAg Level (IU/mL) | HBsAb Level (mIU/mL) |
|---|---|---|---|
| 1 | 3 ug HBsAg | <LOQ* in 1/4 animal, | >LOQ* in 1/4 animal, max individual |
| | | mean = −0.23 log10 | level of 24 mIU/ml |
| 2 | 500 ug | <LOQ in 2/4 animals, | >LOQ in 3/4 animals, geometric mean = |
| | AUS1499 + | mean = −1.10 log10 | 15 mIU/ml, max individual HBsAb level |
| | 3 ug HBsAg | | of 105 mIU/ml |
| 3 | 500 ug | <LOQ in 4/4 animals | >LOQ in 4/4 animals, geometric mean = |
| | AUS1476 + | | 1880 mIU/ml, max individual HBsAb |
| | 3 ug HBsAg | | level of 12728 mIU/ml |
| 4 | 150 ug | <LOQ in 2/4 animals, | >LOQ in 3/4 animals, geometric mean = |
| | AUS1476 + | mean = −1.26 log10 | 9 mIU/ml, max individual level of 67 |
| | 3 HBsAg | | mIU/ml |
| 5 | 500 ug | <LOQ in 4/4 animals | >LOQ in 2/4 animals, geometric mean = |
| | AUS1497 + | | 193 mIU/ml, max individual level of 890 |
| | 3 ug HBsAg | | mIU/ml |
| 6 | 500 ug | <LOQ in 2/4 animals, | >LOQ in 4/4 animals, geometric mean = |
| | AUS1498 + | mean = −0.83 log10 | 25 mIU/ml, max individual HBsAb level |
| | 3 ug HBsAg | | of 1558 mIU/ml |
| 7 | 500 ug | <LOQ in 4/4 animals | >LOQ in 3/4 animals, geometric mean = |
| | AUS1500+ | | 71 mIU/ml, max individual HBsAb level |
| | 3 ug HBsAg | | of 1214 mIU/ml |
| 8 | 500 ug | <LOQ in 4/4 animals | >LOQ in 3/4 animals, geometric mean = |
| | AUS1496 + | | 143 mIU/ml, max individual HBsAb |
| | 3 ug HBsAg | | level of 5669 mIU/ml |

*HBsAg LOQ = 1 IU/mL; HBsAb LOQ = 5 mIU/mL

### Example 3: Immune Enhancing Activity of Oligonucleotides in BALB/c Mice

The immune enhancing activity of oligonucleotides were examined in normal mice, and their ability to boost serum HBsAb antibodies was examined. The oligonucleotides used in this example included reference AUS1233, AUS1443, AUS1444, AUS1458, AUS1459, AUS1460, AUS1461, AUS1462, AUS1463, AUS1464, AUS1465, AUS1466, AUS1467, AUS1468, AUS1469, AUS1470, AUS1471, AUS1472, AUS1473, AUS1474, AUS1475, AUS1476, AUS1477, AUS1478, AUS1479, AUS1480, AUS1488, AUS1489 and AUS1490. The animals used were female BALB/c mice. Normal saline was administered as a negative control. Mixtures of HBsAg and an aluminum adjuvant or CpG1018 (a toll-like receptor 9 (TLR9) agonist adjuvant) were administered as positive controls.

The working solution of each oligonucleotide was prepared in saline at a concentration of 3 mg/ml. The CpG1018 working solution was prepared in saline at 0.3 mg/ml. The aluminum adjuvant (Alum) was purchased from ThermoFisher Scientific, and its working solution was prepared in saline at 0.3 mg/ml. The HBsAg used in this example is a commercially available natural Hepatitis B surface antigen protein (purchased from Beijing Biolab Technology Co., Ltd), which has been isolated and purified from the plasma of chronic Hepatitis B patients. A HBsAg working solution was prepared in saline at a concentration of 0.04 mg/ml.

Female BALB/c mice were randomly divided into groups of 5 mice per group. The dosing solution was prepared by mixing each working solution with the HBsAg working solution at a 1:1 volume ratio approximately 30 min before administering to the animals. Each animal was subcutaneously dosed on Day 0 and Day 14 with 100 μl of a dosing solution containing 2 ug of HBsAg antigen protein and 150 ug of an oligonucleotide or 15 ug CpG1018 or 150 ug Alum adjuvant.

Approximately 100 μl of blood sample was taken from a thigh vein of each animal on Days 7, 14, 21, 28, 35 and 42 post dose. Blood samples were incubated at 37° C. for approximately 30 minutes, and then centrifuged in a precooled (0-4° C.) centrifuged to obtain the serum samples. The serum samples were stored at −20° C. until analysis. HBsAb levels were measured in the serum samples by Chemiluminescence immunoassay assays.

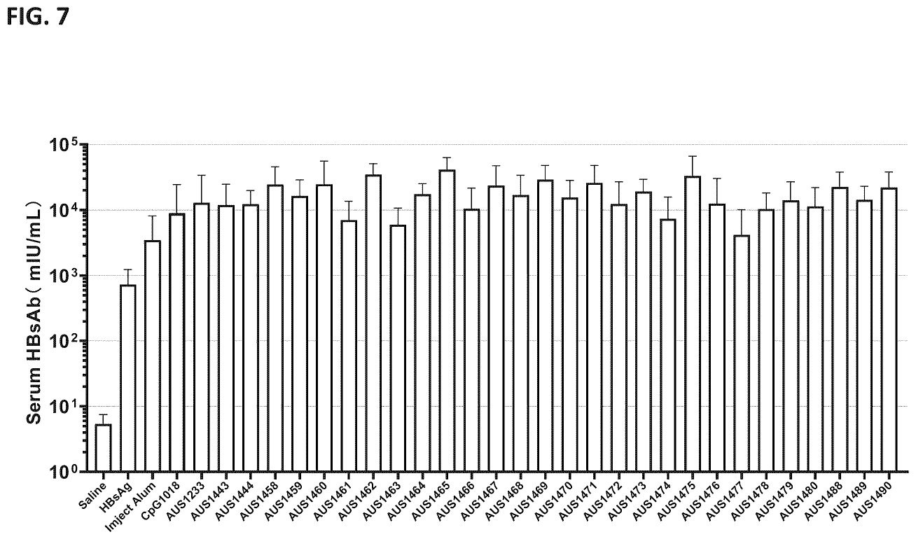

The serum HBsAb levels on Day 42 are listed in Table 3.1 and shown in FIG. 7, the boxes represent the means and the error bars are the standard deviation. The results showed that the immune enhancing activity of the oligonucleotides tested was similar to or better than that of conventional adjuvant Alum and CpG1018 in BALB/c mice, indicating the adjuvant-like properties for oligonucleotides of the disclosure.

TABLE 3
1 Serum HBsAb levels in female BALB/c mice on Day 42

| | | Geometric mean HBsAb |
|---|---|---|
| Group | Dose | Level (mIU/mL) |
| 1 | Saline | |
| 2 | 2 ug HBsAg | 531 |
| 3 | 2 ug HBsAg + Alum | 1883 |
| 4 | 2 ug HBsAg + 15 ug CpG1018 | 2129 |
| 5 | 2 ug HBsAg + 150 ug AUS1233 | 3069 |
| 6 | 2 ug HBsAg + 150 ug AUS1443 | 6243 |
| 7 | 2 ug HBsAg + 150 ug AUS1444 | 6912 |
| 8 | 2 ug HBsAg + 150 ug AUS1458 | 14592 |
| 9 | 2 ug HBsAg + 150 ug AUS1459 | 12841 |
| 10 | 2 ug HBsAg + 150 ug AUS1460 | 5385 |
| 11 | 2 ug HBsAg + 150 ug AUS1461 | 3869 |
| 12 | 2 ug HBsAg + 150 ug AUS1462 | 31625 |
| 13 | 2 ug HBsAg + 150 ug AUS1463 | 3730 |
| 14 | 2 ug HBsAg + 150 ug AUS1464 | 15235 |
| 15 | 2 ug HBsAg + 150 ug AUS1465 | 36307 |
| 16 | 2 ug HBsAg + 150 ug AUS1466 | 4954 |
| 17 | 2 ug HBsAg + 150 ug AUS1467 | 9610 |
| 18 | 2 ug HBsAg + 150 ug AUS1468 | 9520 |
| 19 | 2 ug HBsAg + 150 ug AUS1469 | 19462 |
| 20 | 2 ug HBsAg + 150 ug AUS1470 | 8240 |
| 21 | 2 ug HBsAg + 150 ug AUS1471 | 19247 |
| 22 | 2 ug HBsAg + 150 ug AUS1472 | 4860 |
| 23 | 2 ug HBsAg + 150 ug AUS1473 | 16333 |
| 24 | 2 ug HBsAg + 150 ug AUS1474 | 4714 |
| 25 | 2 ug HBsAg + 150 ug AUS1475 | 14386 |
| 26 | 2 ug HBsAg + 150 ug AUS1476 | 3917 |
| 27 | 2 ug HBsAg + 150 ug AUS1477 | 1298 |
| 28 | 2 ug HBsAg + 150 ug AUS1478 | 4712 |
| 29 | 2 ug HBsAg + 150 ug AUS1479 | 7402 |
| 30 | 2 ug HBsAg + 150 ug AUS1480 | 4781 |
| 31 | 2 ug HBsAg + 150 ug AUS1488 | 12756 |
| 32 | 2 ug HBsAg + 150 ug AUS1489 | 11237 |
| 33 | 2 ug HBsAg + 150 ug AUS1490 | 15811 |

### Example 4: Immune Enhancing Activity of Oligonucleotides in HBV Carrier Mice

The immune enhancing activity of oligonucleotides AUS1476 vs. AUS1233 was evaluated in HDI-HBV mice. The animals were randomly divided into groups of four animals per group. In the pretreatment dosing period, all animals were subcutaneously dosed with 60 mg/kg AUS1233 on Day −37, −30, −23, −16 followed by 60 mg/kg AUS1441 on Day −8 to lower the HBsAg levels. In the second dosing period, the vaccine dosing period, the animals were subcutaneously dosed on Day 0 and Day 14 with 100 μl of a dosing solution comprised of one of the following: saline (Group 1), 3 ug HBsAg (Group 2); a mixture containing 500 ug AUS1476 and 3 ug HBsAg (Group 3); a mixture containing 500 ug AUS1233 and 3 ug HBsAg (Group 4).

The working solution of AUS1476 or AUS1233 was each prepared in saline at 10 mg/ml. The HBsAg used in this example is a commercially available natural Hepatitis B surface antigen protein (purchased from Beijing Biolab Technology Co., Ltd), which has been isolated and purified from the plasma of chronic Hepatitis B patients. The HBsAg working solution was prepared in saline at a concentration of 0.06 mg/ml. The dosing solution for Group 2 was prepared by a two-fold dilution of HBsAg working solution with saline. The dosing solutions for Groups 3 and 4 were prepared by mixing the HBsAg working solution with the 10 mg/ml working solutions of AUS1476 or AUS1233, respectively, at a 1:1 volume ratio approximately 30 min before administering to the animals.

Approximately 100 μl of blood sample was taken from a thigh vein of each animal on Day −37, −30, −23, −16, −8, −1, 7, 14, 21, 28, 35, 42, 49, 56, and 63. Blood samples were incubated at 37° C. for approximately 30 minutes, and then centrifuged in a pre-cooled (0-4° C.) centrifuge to obtain the serum samples. The serum samples were stored in a −20° C. refrigerator until analysis. HBsAg and HBsAb levels were measured in the serum samples by Chemiluminescence immunoassay assays.

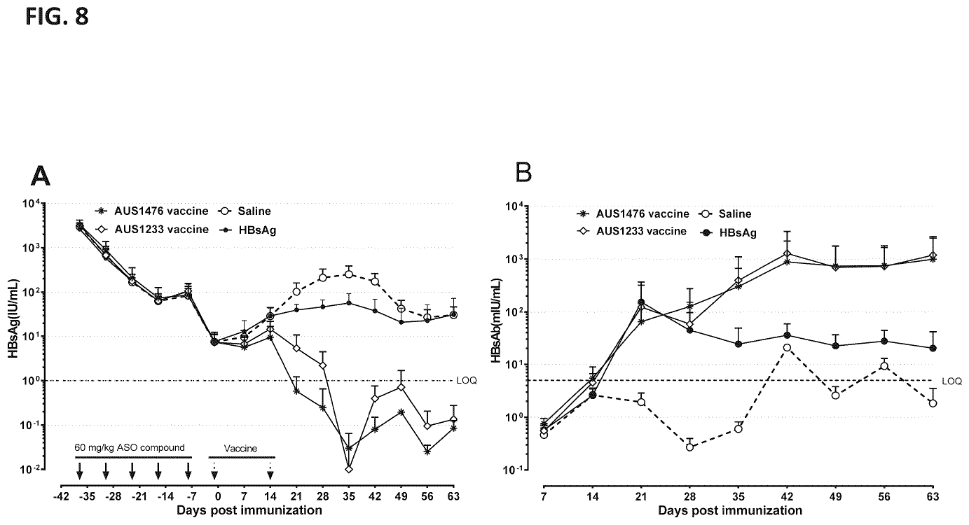

The serum HBsAg levels in the HDI-HBV mice are shown in FIG. 8, Graph A, and the serum HBsAb levels are shown in FIG. 8, Graph B. The data points are the means and error bars are the standard deviation.

The serum levels of HBsAg and HBsAb in HDI-HBV mice on Day 63 are listed in Table 4.1. The combination of AUS1476 and HBsAg (termed AUS1476 vaccine in this example) resulted in significantly stronger and sustained reduction in serum HBsAg levels and induced much higher levels of HBsAb compared to the HBsAg antigen alone. The combination of AUS1233 and HBsAg (termed AUS1233 vaccine in this example) had similar effects on HBsAg and HBsAb levels, although the effects were lower than that achieved by the AUS1476 vaccine.

TABLE 4.1
Serum HBsAg and HBsAb levels in HDI-HBV mice on Day 63

| Group ID | Dose | HBsAg level (IU/mL) | HBsAb level |
|---|---|---|---|
| 1 | Saline | >LOQ in 4/4 animals, | < LOQ in 4/4 animals |
| | | mean = 1.44 log10 | |
| 2 | 3 ug HBsAg | <LOQ in 1/4 animal, | >LOQ in 2/4 animals, geometric |
| | | mean = 0.92 log10 | mean = 9 mIU/ml, max |
| | | | individual HBsAb level of 42 |
| | | | mIU/ml |
| 3 (AUS1476 | 500 ug | <LOQ in 4/4 animals | >LOQ in 4/4 animals, geometric |
| vaccine) | AUS1476 + 3 ug | | mean = 227 mIU/ml, max |
| | HBsAg | | individual HBsAb level of 3174 |
| | | | mIU/ml |
| 4 (AUS1233 | 500 ug | <LOQ in 4/4 animals | >LOQ in 4/4 animals, geometric |
| vaccine) | GSK-836+ 3 ug | | mean = 197 mIU/ml, max |
| | HBsAg | | individual HBsAb level of 3113 |
| | | | mIU/ml |

### Example 5: Immune Enhancing Activity of Oligonucleotides in HBV Carrier Mice

The immune enhancing activity of oligonucleotide AUS1476, with no pretreatment dosing, was evaluated in HDI-HBV mice. The animals were subcutaneously dosed simply with saline on Day 0 and 7, followed by subcutaneous dosing with 100 μl of a mixture containing 500 μg AUS1476 and 3 μg HBsAg on Days 14 and 28.

The AUS1476 working solution was prepared in saline at a concentration of 10 mg/ml. The HBsAg protein used in this example was a commercially available recombinant small Hepatitis B surface antigen protein purchased from Bioforcesci Biomart Co., Ltd., which had been produced and purified from the incubation medium of genetically modified mammalian Chinese hamster ovary (CHO) cells. The HBsAg (CHO) working solution was prepared in saline at a concentration of 0.06 mg/ml. The HBsAg (CHO) working solution was mixed at a 1:1 volume ratio with the AUS1476 working solution approximately 30 min before administering to the animals to make the dosing solution.

Approximately 100 μl of blood sample was taken from a thigh vein of each animal on Day 7, 14, 21, 28, 35, 42, 49, 56, 63, 70 and 77. Blood samples were incubated at 37° C. for approximately 30 minutes, and then centrifuged in a pre-cooled (0-4° C.) centrifuge to obtain the serum samples. The serum samples were stored in a −20° C. refrigerator until analysis. The HBsAg and HBsAb levels in the serum samples were measured by chemiluminescence immunoassay assays.

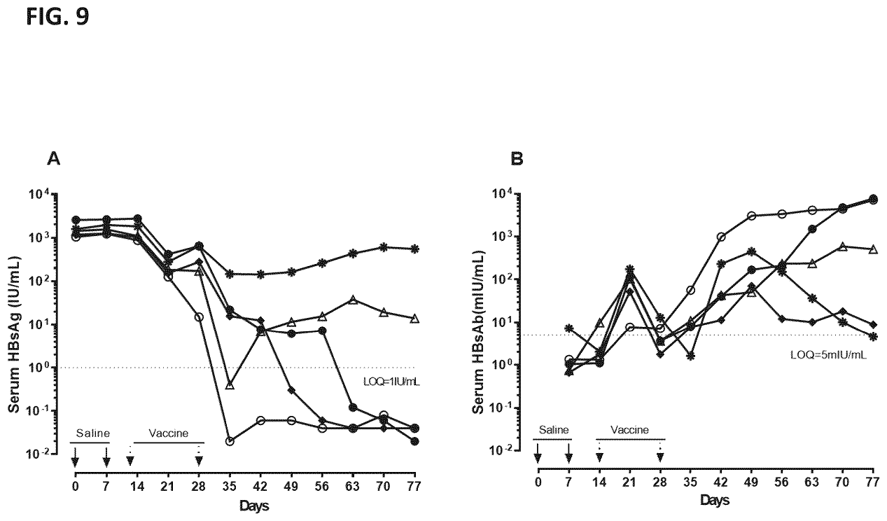

The serum levels of HBsAg and HBsAb in HDI-HBV mice are shown in FIG. 9. The data points in FIG. 9A and FIG. 9B are the individual animal HBsAg or HBsAb levels, respectively. Even though the baseline serum HBsAg levels were high (1044-2570 IU/ml), the mixture of AUS1476 and HBsAg was still able to decrease the serum HBsAg levels to below the limit of quantification (LOQ) in some HDI-HBV mice (3 out of the total 5 animals), and boosted the HBsAb levels to as high as 7694 mIU/ml in these animals.

The results here indicate that a pretreatment dose is optional, prior to the vaccine dosing period, especially if baseline surace antigen levels are low.

### Example 6: Immune Enhancing Effects of Oligonucleotides on the T-Cell Response, and HBV Protein, mRNA and DNA Levels in HBV Carrier Mice

The immune enhancing activity of oligonucleotide AUS1476 was evaluated in HDI-HBV mice, as assayed by HBsAg antigen and HBsAb levels, HBV mRNA and HBV DNA levels, and T-cell response. The animals were dosed with AUS1476 at 40 mg/kg by subcutaneous injection (S.C) in HDI-HBV mice on Day 0, 7, 21, and 35. The animals were also dosed with 100 μl of a mixture containing 500 μg AUS1476 and 3 μg HBsAg protein on Day 14 and 28. The saline control group was dosed with saline only in all treatment period. This mixture is called Vaccine or “AUS1476-enhanced vaccine” in this example.

The AUS1476 dosing solution was prepared in saline at a concentration of 4 mg/ml. The AUS1476 working solution was prepared in saline at a concentration of 10 mg/ml. The HBsAg protein used in this example was a commercially available recombinant small Hepatitis B surface antigen protein purchased from Bioforcesci Biomart Co., Ltd., which had been produced and purified from the incubation medium of genetically modified mammalian Chinese hamster ovary (CHO) cells. The HBsAg (CHO) working solution was prepared in saline at a concentration of 0.06 mg/ml. The vaccine dosing solution was prepared by mixing the HBsAg (CHO) working solution with the AUS1476 working solution at a 1:1 volume ratio approximately 30 min before administering to the animals.

Approximately 100 μl of blood samples were taken from a thigh vein each week. Blood samples were incubated at 37° C. for approximately 30 minutes, and then centrifuged in a precooled (0-4° C.) centrifuge to obtain the serum samples. The serum samples were stored at −20° C. for further analysis. HBsAg and HBsAb levels were measured in the serum samples by Chemiluminescence immunoassay assays.

At the end of the study on Day 77, 30 to 50 mg of liver tissue samples were taken from the animals. Liver tissue DNA samples were prepared using a Total DNA kit from Omega Bio-tek. HBV DNA concentrations in the samples were determined by qPCR. Liver tissue RNA samples were prepared using a Total RNA kit from Omega Bio-tek. HBV RNA concentrations in the samples were determined by RT-qPCR.

Peripheral Blood Mononuclear Cells (PBMC) were isolated from whole blood samples (0.8 ml) collected from the animals at the end of the study on Day 77. Additionally, fresh mouse spleen tissue samples were collected, and splenic T lymphocytes were isolated. HBV-specific T cell response (CD8+/IFNy+%) in the blood PBMC and splenic T lymphocytes were determined by flow cytometer using the following procedure: PBMCs and spleen lymphocyte cells (5×10⁵) were cultured in 200 μl of DMEM medium in the presence or absence of small HBsAg peptide pool (10 ug/mL) in 96-well round-bottom plates. The peptide pool comprises of a total of 50 peptides with small HBsAg specific CTL epitope. Each peptide is 15 amino acids in length with 11 amino acids overlaps between them. After stimulation with the peptide pool for 24 h, the cells were stained with a primary antibody (Anti-Mouse CD3-FITC, Anti-Mouse CD4-PreCP-Cy5.5, Anti-Mouse CD8-APC, or Anti-Mouse IFN-γ-PE) and detected by flow cytometry.

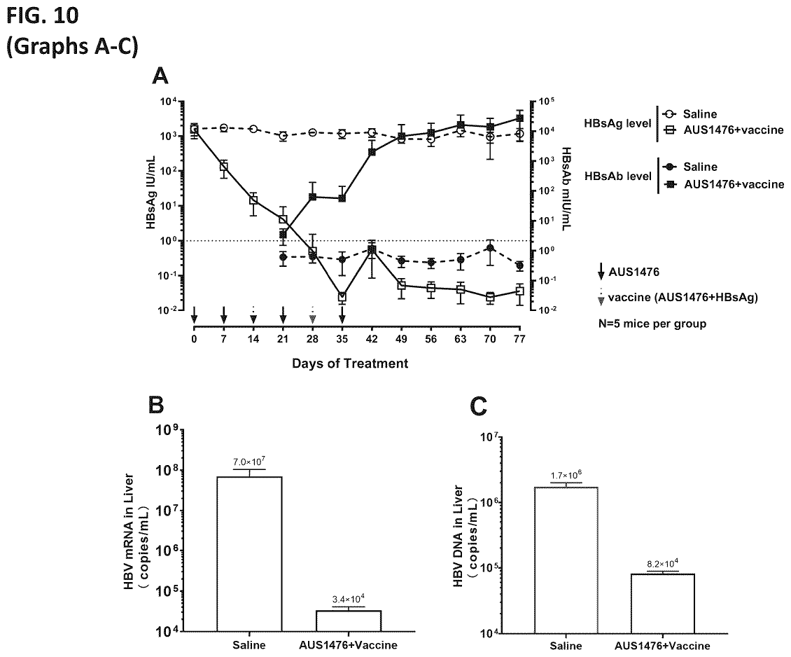

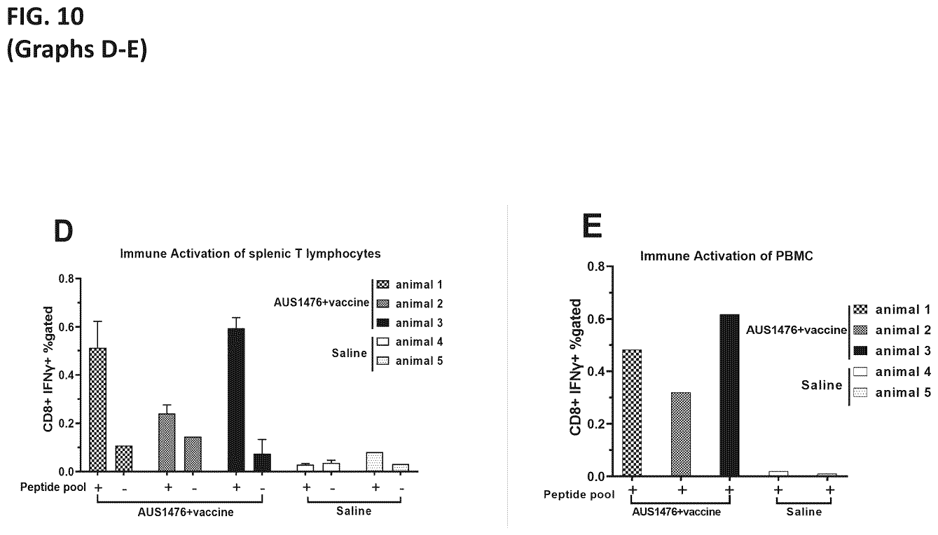

The serum HBsAg and HBsAb levels, T-cell response, HBV mRNA and HBV DNA expression levels on Day 77 are shown in FIG. 10 and Table 6.1. FIG. 10, Graph A shows serum HBsAg and HBsAb levels in HDI-HBV mice. FIG. 10, Graph B and C show HBV mRNA and HBV DNA levels in the liver. FIG. 10, Graph D shows immune activation of spleen T lymphocytes. FIG. 10, Graph E shows the immune activation of PBMCs. The data points are the means and error bars are the standard deviation. In FIG. 10 dosing with the combination of AUS1476 alone is designated as “AUS1476” alone; and dosing with AUS1476 in combination HBsAg is designated as “AUS1476+vaccine”.

Dosing with the combination of AUS1476 alone, or with AUS1476 (500 ug) in combination with 3 ug HBsAg (the vaccine composition) achieves sustained HBsAg clearance in HDI-HBV mice, with strong anti-HBs response (HBsAb), a marked reduction of HBV-positive cells in the liver as indicated by the significant decrease of liver HBV mRNA and HBV DNA levels. The flow cytometry results in splenic lymphocytes and PBMC cells further demonstrated efficient induction of HBV specific T cell immune response for elimination of HBV-positive hepatocytes.

TABLE 6.1
Effect of the AUS1476-enhanced vaccine in HDI-HBV mice, Day 77 data

| Index | Saline | AUS1476-enhanced vaccine |
|---|---|---|
| HBsAg in serum | No activity | <LOQ* in 4/4 animals |
| HBsAb in serum | No activity | >LOQ* in 4/4 animals, geometric mean = 30000 |
| | | mIU/ml |
| HBV mRNA in liver | No activity | Compared to saline, decrease more than 3 log10 |
| HBV DNA in liver | No activity | Compared to saline, decrease more than 1.3 log10 |
| CD8+/IFNγ+ % gated | No activity | 3/3 mice have HBV specific CTL response |
| in spleen PBMC | | |
| CD8+/IFNγ+ % gated | No activity | 3/3 mice have HBV specific CTL response |
| in blood PBMC | | |

*HBsAg LOQ-1 IU/ml; HBsAb LOQ = 5 mIU/ml

### Example 7: Immune Enhancing Activity of Oligonucleotides in Primates

The immune enhancing activity of an oligonucleotide AUS1476 on the serum HBsAb levels was evaluated in Rhesus monkeys. The animals were dosed by subcutaneous injection (S.C) on Days 0 and 14 with a AUS1476-enhanced vaccine (AUS1476 vaccine), which consisted of 6 μg HBsAg and either 10 mg or 40 mg of AUS1476. As a positive control, the CpG1018 vaccine consisted of 6 ug HBsAg and 0.6 mg CpG1018, and was dosed in a separate group of animals by intramuscular injection (I.M) on Days 0 and 14. The serum HBsAb levels were measured in the serum samples by Chemiluminescence immunoassay assays.

The AUS1476 working solutions were prepared in saline at a concentration of 8 mg/ml or 32 mg/ml. CpG1018 working solution was prepared in saline at a concentration of 2.4 mg/ml. The HBsAg protein used in this example was a commercially available recombinant small Hepatitis B surface antigen protein purchased from Bioforcesci Biomart Co., Ltd., which had been produced and purified from the incubation medium of genetically modified mammalian Chinese hamster ovary (CHO) cells. HBsAg working solution was prepared in saline at a concentration of 24 ug/ml.

The dosing solution of the AUS1476-enhanced vaccine (AUS1476 Vaccine) was prepared by mixing 250 μl of HBsAg working solution with 1250 μl of AUS1476 working solution approximately 30 min before dosing the animals. Each animal was subcutaneously injected with 1.5 ml of the AUS1476 Vaccine dosing solution. The dosing solution of the CpG1018 vaccine was prepared by mixing 250 μl of HBsAg working solution with 250 μl of CpG1018 working solution approximately 30 min before dosing the animals. Each animal was intramuscularly injected with 0.5 ml of the CpG1018 vaccine dosing solution.

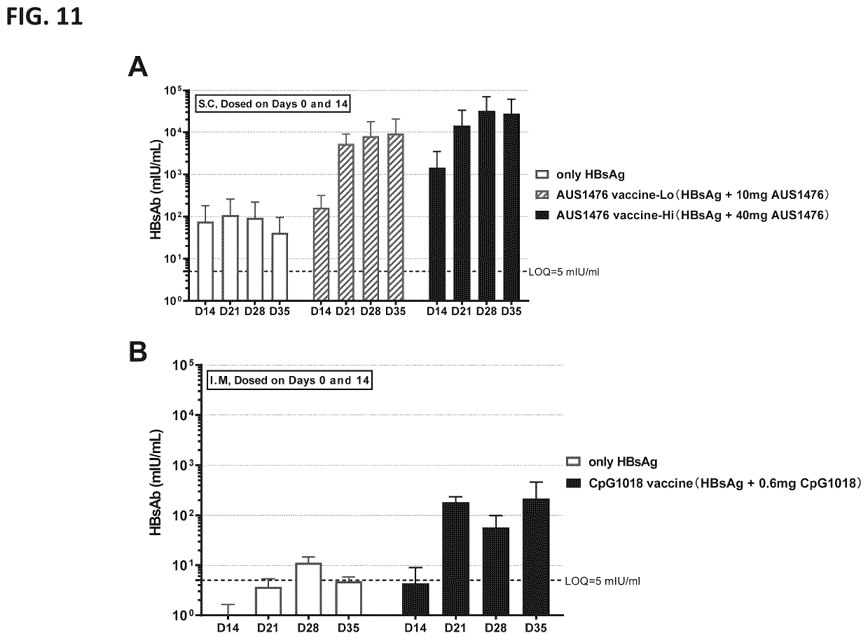

The serum HBsAb (mIU/mL) levels on Days 14, 21, 28, and 35 are shown in FIG. 11. The data points are the means and error bars are the standard deviation. The serum HBsAb (mIU/mL) levels on Day 35 are listed in Table 7. 1. AUS1476-enhanced vaccine induced high and persistent levels of anti-HBs antibody in a dose dependent manner, demonstrating that oligonucleotide compound AUS1476 functioned as an immune activator to induce strong anti-HBsAg antibody response in monkeys. In contrast, TLR9 adjuvant CpG1018 vaccine induced much lower level and less persistent anti-HBs antibody response.

TABLE 7.1
Serum HBsAb levels in Rhesus monkey on Day 35

| | Dosing | HBsAb Level |
|---|---|---|
| Group | Route | (mIU/ml, Geometric mean) |
| HBsAg Only | S.C. | 15 |
| AUS1476 vaccine-Lo (10mg) | S.C. | 4750 |
| AUS1476 vaccine-Hi (40mg) | S.C. | 13323 |
| HBsAg Only | I.M. | 5 |
| CpG1018 vaccine (0.6 mg) | I.M. | 126 |

### Example 8: Antibody Induction Kinetics of Oligonucleotides

The immune enhancing activity of oligonucleotides of the disclosure, on the serum HBsAb levels was evaluated in female BALB/c mice. The oligonucleotides used study included AUS1476 and AUS1500.

The working solutions of the oligonucleotide compounds were prepared in saline at a concentration of 3 mg/ml. The working solution of CpG1018 was prepared in saline at a concentration of 0.3 mg/ml. The aluminum adjuvant (Alum) was purchased from ThermoFisher Scientific, and its working solution was prepared in saline at 3 mg/ml. The HBsAg used in this example was a commercially available natural Hepatitis B surface antigen protein (purchased from Beijing Biolab Technology Co., Ltd), which has been isolated and purified from the plasma of chronic Hepatitis B patients. HBsAg working solution was prepared in saline at a concentration of 0.04 mg/ml.

The dosing solutions were prepared by mixing equal volumes of the HBsAg working solution with each of the working solutions of the oligonucleotide compounds or the CpG1018 working solution approximately 30 min before administering to the animals. The dosing solution for the Alum adjuvanted HBsAg was prepared by adding and mixing the aluminum adjuvant working solution to the HBsAg working solution at a volume ratio of 1:1.

The animals were randomly divided into groups of 5 animals per group. Each group of animals was subcutaneously dosed with a 100 μl of a dosing solution on Days 0 and 14.

Approximately 100 μl of blood sample was taken from a thigh vein of the animals on Days 14, 21, 28, 35 and 42 post dose. Blood samples were incubated at 37° C. for approximately 30 minutes, and then centrifuged in a precooled (0-4° C.) centrifuge to obtain the serum samples. The serum samples were stored at −20° C. until analysis. HBsAb levels were measured in the serum samples by Chemiluminescence immunoassay assays.

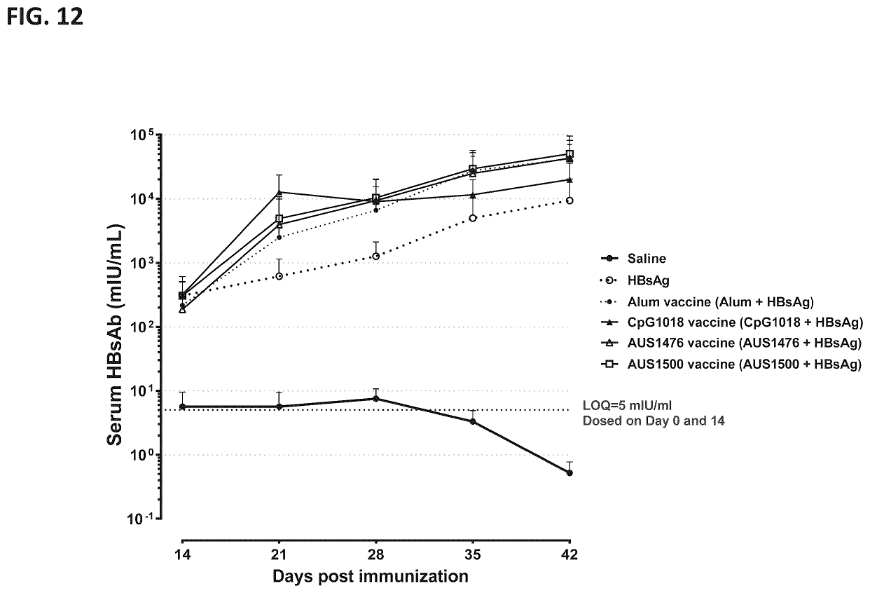

The serum HBsAb (mIU/mL) levels as a function of time are shown in FIG. 12. The data points are the means and the error bars are the standard deviation. The serum HBsAb levels on Day 42 are listed in Table 8.1. The results showed that the oligonucleotide-enhanced vaccines had a different HBsAb induction kinetics from the CpG1018 adjuvanted HBsAg. The HBsAb level increased faster following CpG018 adjuvanted vaccine, reaching peak level 7 days after the second dose of the vaccine, then plateaued. In contrast, the HBsAb level following oligonucleotide-enhanced vaccines increased steadily over time.

TABLE 8.1
Serum HBsAb levels in female BALB/c mice on Day 42

| Group | HBsAb (Geometric mean) |
|---|---|
| Saline | <LOQ in 4/4 animals |
| HBsAg (2 ug) | 4932 mIU/ml |
| 150 ug Alum + 2 ug HBsAg | 34789 mIU/ml |
| 15 ug CpG1018 + 2 ug HBsAg | 12930 mIU/ml |
| 150 ug AUS1476 + 2 ug HBsAg | 22144 mIU/ml |
| 150 ug AUS1500 + 2 ug HBsAg | 40796 mIU/ml |

### Example 9: Effect of Premixing the Oligonucleotide with the HBsAg on the Immune Enhancing Effects

The immune enhancing activity of the oligonucleotides, when subcutaneously dosed along with HBsAg either as a pre-mixed dosing solution or as two separate injections to adjacent sites, was evaluated in female BALB/c mice. The oligonucleotides used in this study included AUS1233, AUS1441, AUS1462 and AUS1476.

Female BALB/c mice were randomly divided 6 mice per group. The animals were subcutaneously dosed on Days 0 and 14 with either 100 μl of the pre-mixed dosing solution or 50 μl of the HBsAg working solution followed by 50 μl of an oligonucleotide working solution. In either case, each dose contains 150 ug oligonucleotide and 2 ug HBsAg protein. Normal saline was administered as negative control, and HBsAg only was administered as positive control. The HBsAb levels were measured in the serum samples by chemiluminescence immunoassay assays.

The working solution of each oligonucleotide compound was prepared in saline at a concentration of 3 mg/ml. The HBsAg working solution was prepared in saline at 0.04 mg/ml. The HBsAg used in this example is a commercially available natural Hepatitis B surface antigen protein (purchased from Beijing Biolab Technology Co., Ltd), which has been isolated and purified from the plasma of chronic Hepatitis B patients. When dosed as a pre-mixed mixture, the dosing solutions were prepared mixing equal volumes of the HBsAg working solution with the working solution of each oligonucleotide approximately 30 min before administering to the animals.

Approximately 100 μl of blood sample was taken from a thigh vein of each animal on Day 14, 21, 28, 35 and 42. Blood samples were incubated at 37° C. for approximately 30 minutes, and then centrifuged in a precooled (0-40 C) centrifuge to obtain the serum samples. The serum samples were stored in a −20° C. until analysis. The serum HBsAb levels was measured in the serum samples by Chemiluminescence immunoassay assays.

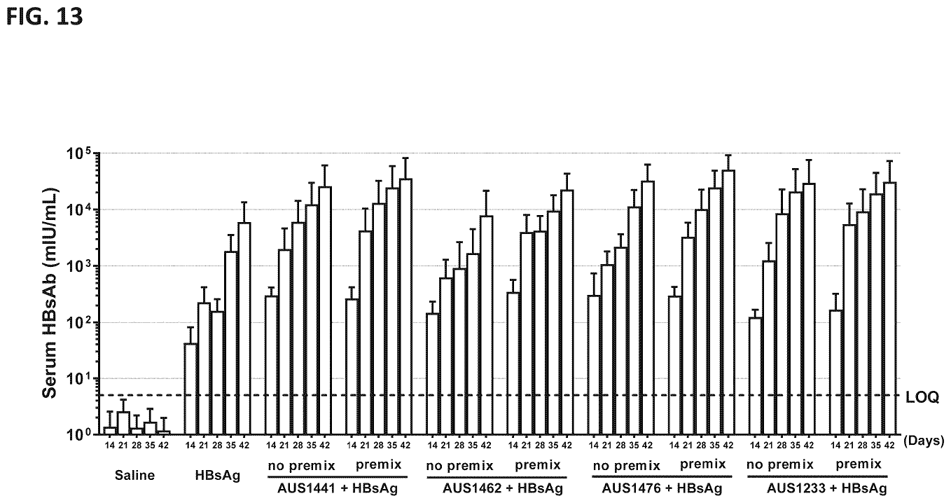

The serum HBsAb (mIU/mL) levels in female BALB/c mice on Days 14, 21, 28, 35 and 42 are shown in FIG. 13. The boxes are the means and error bars are the standard deviation. The serum HBsAb (mIU/mL) levels on Day 42 are listed in Table 9.1. The results showed different kinetics of HBsAb production when the oligonucleotides were administered with HBsAg either as a pre-mixed dosing solution or as separate injections at adjacent injection sites. In early time period, HBsAb levels were higher with pre-mixed dosing solutions. But in later time period, similar HBsAb levels were obtained with either method of dosing. The results show that dosing oligonucleotides and the antigen as separate injections at adjacent injection sites is a feasible alternative to dosing them as pre-mixed dosing solutions.

TABLE 9.1
Serum HBsAb levels (mIU/ml )in female BALB/c mice on Day 42

| | No premix dosing | Premix dosing |
|---|---|---|
| Group | Geometric Mean (CV %) | Geometric Mean (CV %) |
| Saline | Not applicable | <LOQ, LOQ = 5 mIU/ml |
| HBsAg (CHO) | Not applicable | 3670 (122%) |
| AUS1441 | 7988 (135%) | 7646 (127%) |
| AUS1462 | 3294 (172%) | 11369 (90%) |
| AUS1476 | 22468 (92%) | 29505 (81%) |
| AUS1233 | 6154 (155%) | 11427 (134%) |

### Example 10: Immune Enhancing Activity of Oligonucleotide Compounds in BALB/c Mice

The immune enhancing activities of oligonucleotides including AUS1476, AUS1233, AUS1193, AUS1194, and AUS1709 were evaluated in female BALB/c mice. Normal saline was administered as a negative control. The AUS1709 sequence is not complementary to any portion of the nucleic acid sequence of a HBV genome.

The working solution of each oligonucleotide was prepared in saline at a concentration of 6 mg/ml. The HBsAg protein used in this example was a commercially available recombinant small Hepatitis B surface antigen protein purchased from Bioforcesci Biomart Co., Ltd., which had been produced and purified from the incubation medium of genetically modified mammalian Chinese hamster ovary (CHO) cells. HbsAg working solution was prepared in saline at a concentration of 0.04 mg/ml.

Female BALB/c mice were randomly divided into groups of 3 to 5 mice per group. The dosing solution was prepared by mixing equal volume of the working solution of each oligonucleotide or positive control with the HBsAg working solution approximately 30 min before administering to the animals. Each animal was subcutaneously dosed on Day 0 and Day 14 with 100 μl of a dosing solution containing 2 ug of HBsAg antigen protein and 300 ug of an oligonucleotide.

Approximately 100 μl of blood sample was taken from a thigh vein of the animals on Days 14 and 21 post-dose. Blood samples were incubated at 37° C. for approximately 30 minutes, and then centrifuged in a precooled (0-4° C.) centrifuge to obtain the serum samples. The serum samples were stored at −20° C. until analysis. HBsAb levels were measured in the serum samples by Chemiluminescence immunoassay assays.

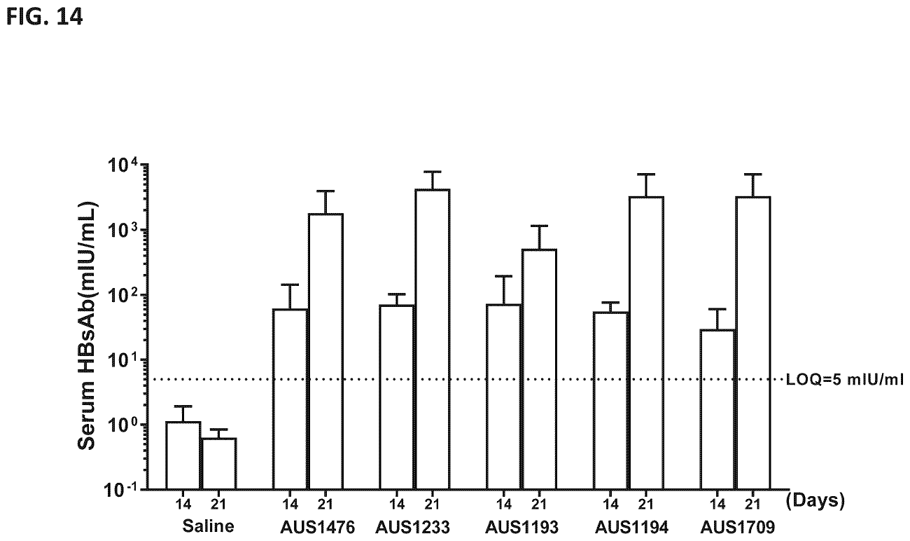

The serum HBsAb levels on Day21 are listed in Table 1 and shown in FIG. 14. The boxes represent the means and the error bars are the standard deviation. The results show that the immune enhancing activity of the oligonucleotides does not depend on complementarity to a portion of the nucleic acid sequence of a HBV genome.

TABLE 10.1
Serum HBsAb levels in female BALB/c mice on Day 21

| Group | | |
|---|---|---|
| ID | Dose | Geometric mean HBsAb Level |
| 1 | Saline | — |
| 2 | 2 ug HBsAg + | Geomean HBsAb level of 676 mIU/ml, |
| | 300u g | max individual HBsAb level of 4253 |
| | AUS1476 | mIU/ml |
| 3 | 2 ug HBsAg + | Geomean HBsAb level of 3149 mIU/ml, |
| | 300 ug | max individual HBsAb level of 6099 |
| | AUS1233 | mIU/ml |
| 4 | 2 ug HBsAg + | Geomean HBsAb level of 151 mIU/ml, |
| | 300 ug | max individual HBsAb level of 1572 |
| | AUS1193 | mIU/ml |
| 5 | 2 ug HBsAg + | Geomean HBsAb level of 1441 mIU/ml, |
| | 150 ug | max individual HBsAb level of 9346 |
| | AUS1194 | mIU/ml |
| 6 | 2 ug HBsAg + | Geomean HBsAb level of 2093 mIU/ml, |
| | 150 ug | max individual HBsAb level of 17874 |
| | AUS1709 | mIU/ml |

### Example 11: Immune Enhancing and Antiviral Activity of Administering Oligonucleotide Followed by a Vaccine in a HBV Model

The immune enhancing and antiviral activities of the oligonucleotide AUS1476 and a recombinant small HBsAg with aluminum hydroxide adjuvant (vaccine) were tested when administered to HBV mice alone or sequentially. Normal saline was administered as a negative control.

Recombinant adeno-associated virus carrying 1.3 copies of hepatitis B virus genome (rAAV-HBV1.3-mer WT replicon) were used, and stored at −70° C. before use. Male C57BL/6 mice aged 4-5 weeks were used to construct the AAV-HBV mouse model (rAAV-1.3HBV-GTD carrier mice) by hydrodynamic-injection of the recombinant adeno-associated virus. HBsAg-positive mice were used 57 days after the injection. The mice were maintained under specific pathogen-free conditions in a BSL-2+ animal facility.

The AUS1476 dosing solution was prepared by dissolving the compound in saline to make a stock solution at 10 mg/ml, which was diluted with saline again at a target concentration of 4.0 mg/ml. The HBV antigen used was a commercially available vaccine from NCPC Genetech Biotechnology (China), which is made up of a recombinant small HBsAg that is produced in mammalian Chinese hamster ovary (CHO) cells, and an aluminum hydroxide adjuvant. The concentration of the recombinant small HBsAg plus alum working solution was 20 ug/ml.

Fifteen male rAAV-1.3HBV-GTD carrier mice were divided into four group of 3 to 5 mice, as shown in Table 11.1. In Group 1, the animals were administered saline on Day 1, 4, 8, 11, 18, 32, and 46. In Group 2, the animals were administered saline on Day 1, 4, 8, and 11, followed by three doses (3 ug per animal) of alum-adjuvanted recombinant small HBsAg via intramuscular injection on Day 18, 32, and 46. In Group 3, the animals were administered AUS1476 at 40 mg/kg subcutaneously on Day 1, 4, 8, and 11, followed by three doses of saline on Day 18, 32, and 46. In Group 4, the animals were administered AUS1476 at 40 mg/kg subcutaneously on Day 1, 4, 8, and 11, followed by three doses (3 ug per animal) of alum-adjuvanted recombinant small HBsAg via intramuscular injection on Day 18, 32, and 46.

TABLE 11.1
Dosing schedule for AUS1476 and HBsAg + alum adminsitration to mice

| Group | | No. of | | | | | | | |
|---|---|---|---|---|---|---|---|---|---|
| ID | Dose | mice | Day 1 | Day 4 | Day 8 | Day 11 | Day 18 | Day 32 | Day 46 |
| 1 | Saline | 3 | Saline | Saline | Saline | Saline | Saline | Saline | Saline |
| 2 | 3 ug | 3 | Saline | Saline | Saline | Saline | HBsAg + | HBsAg + | HBsAg + |
| | HBsAg + | | | | | | alum | alum | alum |
| | alum | | | | | | | | |
| 3 | 40 mg/kg | 4 | AUS1476 | AUS1476 | AUS1476 | AUS1476 | Saline | Saline | Saline |
| | AUS1476 | | | | | | | | |
| 4 | 40 mg/kg | 5 | AUS1476 | AUS1476 | AUS1476 | AUS1476 | HBsAg + | HBsAg + | HBsAg + |
| | AUS1476 + | | | | | | alum | alum | alum |
| | 3 ug | | | | | | | | |
| | HBsAg + | | | | | | | | |
| | alum | | | | | | | | |

Approximately 100 μl of blood sample was taken from a thigh vein on Day 1 (before dosing), and 4, 8, 11, 15, 18, 25, 32, 39, 46 and 52 days after dosing. Blood samples were placed in an incubator at 37° C. for approximately 30 minutes, and then centrifuged in a precooled centrifuge (0-4° C.) to obtain the serum samples. The obtained serum samples were stored in a −20° C. refrigerator for further analysis.

Serum hepatitis B surface antigen (HBsAg) and hepatitis B surface antibody (HBsAb) levels were measured using chemiluminescence immunoassays.

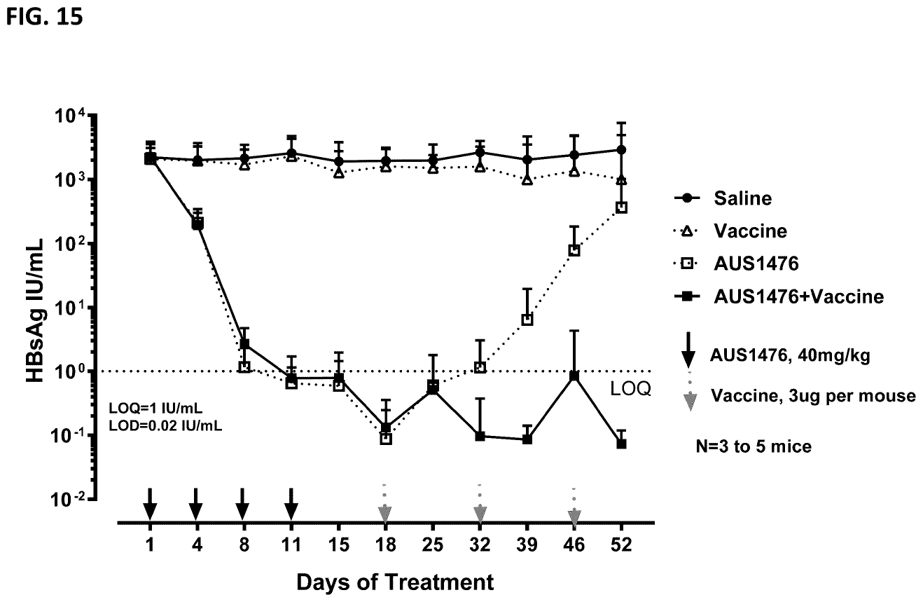

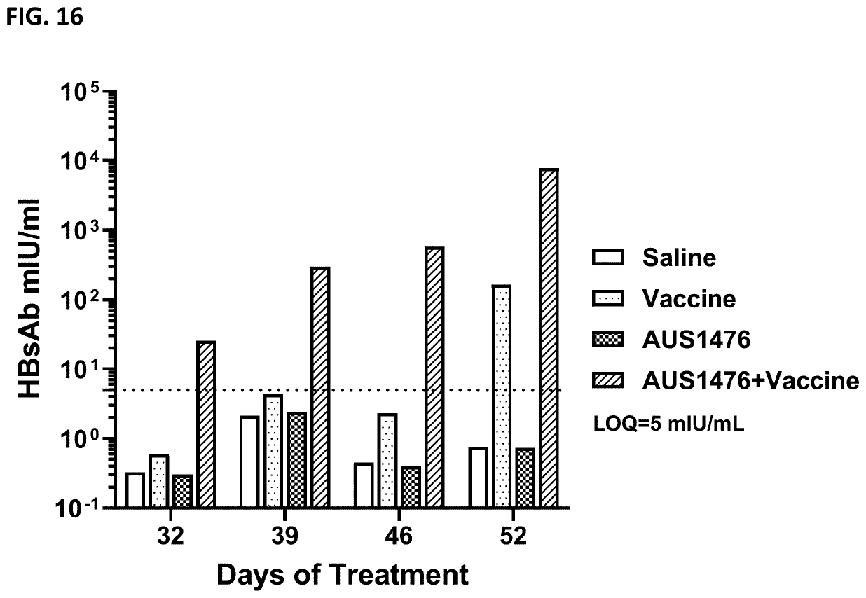

The serum levels of HBsAg and HBsAb in AAV-HBV mice are shown in Table 11.2 and FIGS. 15-16. In FIGS. 15-16, alum-adjuvanted recombinant small HBsAg is referred to as “vaccine.”

TABLE 11.2
Serum HBsAg and HBsAb levels in AAV-HBV mice during the treatment

| Group | | | |
|---|---|---|---|
| ID | Dose | HBsAg reduction | HBsAb level on Day 52 |
| 1 | Saline | No change | Undetectable |
| 2 | 3 ug HBsAg + alum | Reduced by 1 log10 in 1/3 | 2/3 animals produced HBsAb |
| | | animal; no change in 2/3 | and up to 2867 mIU/mL in |
| | | animal | one animal |
| 3 | 40 mg/kg AUS1476 | Reduced to lower limit of | Undetectable |
| | | quantification (LLOQ) but | |
| | | rebounded in 4/4 animals | |
| 4 | 40 mg/kg AUS1476 + | Reduced to LLOQ and | 5/5 animals produced HBsAb |
| | 3 ug HBsAg + alum | sustained in 5/5 animals | and the level was 603 to |
| | | | 26622 mIU/mL |

In the group treated with AUS1476 alone (Group 3), serum HBsAg reduction below the lower limit of quantification of 1 IU/mL was achieved in all four animals after four doses of AUS1476, but the serum HBsAg level rebounded to approximately the baseline level by Day 52 (FIG. 15). No animal produced detectable HBsAb in serum in this group (FIG. 16).

In the group treated with alum-adjuvanted recombinant small HBsAg alone (Group 2), just one of the three animals had HBsAg levels decreased by 1 log 10. Serum HBsAb was detectable in this group, but the HBsAb levels were generally low and variable; the highest one was merely 2867 mIU/ml on Day 52.

In contrast, in the group treated first with AUS1476 and subsequently with alum-adjuvanted recombinant small HBsAg (Group 4), sustained serum HBsAg reduction below the lower limit of 1 IU/ml was achieved in all five animals tested (FIG. 15). Serum HBsAb levels were boosted in all animals in this group, which reached up to 7796 mIU/ml (geometric mean) on Day 52 (FIG. 16).

In summary, administering the oligonucleotide AUS1476 followed by an alum-adjuvanted recombinant small HBsAg effectively and sustainably reduced serum HBsAg and promoted HBsAb production in AAV-HBV mice. Therefore, sequential administration of an oligonucleotide followed by an HBV antigen may be effective for treating and preventing HBV infection.

While the inventions have been described with reference to the specific embodiments thereof it should be understood by those skilled in the art that various changes may be made and equivalents may be substituted without departing from the true spirit and scope of the inventions. In addition, many modifications may be made to adopt a particular situation, material, composition of matter, process, process step or steps, to the objective spirit and scope of the described inventions. All such modifications are intended to be within the scope of the claims appended hereto.

Patents, patent applications, patent application publications, journal articles and protocols referenced herein are incorporated by reference in their entireties, for all purposes.

1. A vaccine composition comprising at least one oligonucleotide and at least one hepatitis B virus (HBV) antigen.

2. The vaccine composition of claim 1, wherein the oligonucleotide is complementary to one or more portions of the nucleic acid sequence of a HBV genome.

3. The vaccine composition of claim 1, wherein the oligonucleotide is not complementary to a portion of the nucleic acid sequence of a HBV genome.

4. The vaccine composition of claim 2, wherein the HBV genome is the genome of HBV GT-A, GT-B, GT-C, GT-D, GT-E, GT-F, GT-G, GT-H, GT-I, or GT-J.

5. The vaccine composition of claim 1, wherein the oligonucleotide comprises 10-60 nucleobases.

6. The vaccine composition of claim 1, wherein the oligonucleotide is a modified oligonucleotide.

7. The vaccine composition of claim 1, wherein the modified oligonucleotide comprises one or more modified sugars, modified bases, modified internucleoside linkages, or combinations thereof.

8. The vaccine composition of claim 1, wherein at least one internucleoside linkage is a phosphodiester linkage.

9. The vaccine composition of claim 1, wherein at least one internucleoside linkage is a modified internucleoside linkage.

10. The vaccine composition of claim 9, wherein the modified internucleoside linkage is a phosphorothioate internucleoside linkage.

11. The vaccine composition of claim 1, wherein the oligonucleotide comprises linked deoxynucleosides.

12. The vaccine composition of claim 1, wherein the oligonucleotide comprises at least one nucleoside comprising a modification to a C2′ position.

13. The vaccine composition of claim 12, wherein the oligonucleotide comprises at least one nucleoside comprising a 2′-O-methyl sugar modification, 2′-O-methoxyethyl sugar modification, 2′-fluoro sugar modification, and/or a 2′-fluoro-arabinonucleic acid (2′-fluoro-ANA) sugar modification.

14. The vaccine composition of claim 1, wherein the oligonucleotide comprises at least one bicyclic sugar modified nucleoside.

15. The vaccine composition of claim 1, wherein the oligonucleotide comprises at least one nucleoside comprising a phosphorodiamidate morpholino modification.

16. The vaccine composition of claim 1, wherein the oligonucleotide comprises at least one locked nucleic acid (LNA), peptide nucleic acid, and/or glycol nucleic acid.

17. The vaccine composition of claim 1, wherein the oligonucleotide is selected from SEQ ID NO: 10-666, or an oligonucleotide having at least 1 sequence modification thereto.

18. The vaccine composition of claim 1, wherein the oligonucleotide is selected from SEQ ID NO: 10-666, or an oligonucleotide having at least 70% sequence identity thereto.

19. The vaccine composition of claim 1, comprising at least two different oligonucleotides.

20. The vaccine composition of claim 1, wherein the HBV antigen is a human HBV antigen.

21. The vaccine composition of claim 1, wherein the HBV antigen is a HBV surface antigen (HBsAg), HBV e antigen (HBeAg), HBV core antigen (HBcAg), or any combination thereof.

22. The vaccine composition of claim 21, wherein the HBsAg is a Small-HBsAg, Medium-HBsAg, Large-HBsAg, or any combination thereof.

23. The vaccine composition of claim 1, wherein the HBV antigen is from a HBV GT-A, GT-B, GT-C, GT-D, GT-E, GT-F, GT-G, GT-H, GT-I, or GT-J genotype.

24. The vaccine composition of claim 1, wherein the HBV antigen is isolated from patients, recombinantly produced, or synthesized.

25. The vaccine composition of claim 1, comprising at least two different HBV antigens.

26. The vaccine composition of claim 1, wherein the vaccine composition comprises an adjuvant.

27. The vaccine composition of claim 26, wherein the adjuvant is an aluminum hydroxide, a toll-like receptor 9 (TLR9) agonist, an oligonucleotide, or a CpG1018.

28-30. (canceled)

31. The vaccine composition of claim 1, wherein the vaccine composition is a therapeutic vaccine composition.

32. The vaccine composition of claim 1, wherein the vaccine composition is a prophylactic vaccine composition.

33. A pharmaceutical composition comprising the vaccine composition of claim 1, and a pharmaceutically acceptable carrier, diluent, or excipient.

34. A kit comprising the vaccine composition of claim 1.

35. A method of eliciting an immune response to a HBV antigen in a subject suffering from an HBV infection, or a HBV-related disease, disorder, or condition, comprising administering to the subject a vaccine composition of claim 1.

36. A method of treating or preventing an HBV infection, or a HBV-related disease, disorder, or condition in a subject with a vaccine treatment, comprising administering to the subject a vaccine composition of claim 1.

37. A method of eliciting an immune response to a HBV antigen in a subject suffering from an HBV infection, or a HBV-related disease, disorder, or condition with a vaccine treatment, comprising administering to the subject an oligonucleotide and a hepatitis B virus (HBV) antigen

38. A method of treating or preventing an HBV infection, or a HBV-related disease, disorder, or condition in a subject with a vaccine treatment, comprising administering to the subject an oligonucleotide and a hepatitis B virus (HBV) antigen.

39-75. (canceled)

76. A kit comprising an oligonucleotide and a hepatitis B virus (HBV) antigen.
